(19) United States
(12) Patent Application Publication
(10) Pub. No.: US 2003/0216974 A1
(43) Pub. Date: Nov. 20, 2003

(54) System and method for drug sample inventory and tracking

(76) Inventor: Richard Browne, Kankakee, IL (US)

Correspondence Address: QUARLES & BRADY LLP, CITICORP CENTER, 500 WEST MADISON STREET, SUITE 3700, CHICAGO, IL, 60661-2511, US

(21) Appl. No.: 10/236,898

(22) Filed: Sep. 6, 2002

Related U.S. Application Data
(60) Provisional application No. 60/381,985, filed on May 17, 2002.

(51) Int. Cl. G06F 17/60

(52) U.S. Cl. 705/28; 705/2

(57) ABSTRACT

A medical center computer system, located at a remote medical center, is used to enter and store information regarding the receipt, dispensing, and inventory of drug samples, automatically check the expiration date of drug samples received and dispensed, enter and store information regarding patients to which drug samples are dispensed, and to automatically enter drug sample receipt information into patient charts. The drug sample receipt, dispensing, and inventory information, and possibly patient information, are then communicated to and stored on a central server. The drug sample receipt, dispensing, and inventory information, and possibly patient information, relating to drug samples from a particular drug company are then communicated to a drug company computer system for the particular drug company, located at a remote drug company facility, where the drug company can access the information.

### CROSS-REFERENCE TO RELATED APPLICATIONS

This application claims priority of U.S. Provisional Patent Application No. 60/381,985, filed May 17, 2002.

### BACKGROUND OF THE INVENTION

When drug samples are received from drug companies at medical centers, such as physician offices and hospitals, the drug samples are typically tracked and inventoried using paper driven systems, if they are tracked at all. Under the current paper driven systems, a drug company representative will visit a medial office, meet with a physician, discuss the drug samples available, and leave various drug samples with the physician. The physician (or his/her clinic) must first manually check each drug sample received to make sure it has not expired, store the drug samples in what is commonly referred to as a “drug closet”, and must keep track of the inventory of the drug closet in some manner. In addition, physicians also usually have some type of organizational plan to “rotate stock” or some other method to maintain the sample closet to avoid having medicines expire on the shelf and to avoid dispensing expired medications. Finally, drug company representatives must record and keep track of the drug samples they have delivered to each medical center that they visit. Both medical centers and drug company representatives typically keep these inventory and organization records by keeping paper records. These tracking and inventory methods are ineffective and time consuming and generally with these systems there is poor accountability and compliance with regulations and regulatory bodies.

When a physician dispenses drug samples to a patient, he must first check to verify that the particular drug sample has not expired by reviewing printed information that may be on the sample itself or by checking the paperwork received from the representative. The physician then must fill out a variety of paper forms to keep track of the samples once the samples are removed from the drug closet to keep track of the inventory and location of the drug samples. Such forms may require information such as the patient's name the drug sample was dispensed to, the name of drug company, the name of the drug, the strength of drug, the number of drug samples dispensed, the date the samples were dispensed, the lot number, the NDC number, the expiration date, and possibly the directions for how the drug sample is to be taken. In addition to this paperwork, much of this same information must also be recorded in the patient's chart and in a logbook to track each drug sample. This environment is very frustrating for physicians and may lead to the physician refusing to accept drug samples at all.

In addition, the current systems and methods are even more ineffective, inaccurate, and time consuming for drug company representatives. When a drug company representative delivers a drug sample to a medical center, the drug company representatives has to either fill out paper forms that carry preprinted information on each of the drug samples delivered, recording information such as the name of the drug sample manufacturer, the name of the drug sample, the strength of the drug sample, the number of samples within a container, the number of containers delivered, the lot number, the NDC number, the expiration date, and the date sample is delivered, and receive a signature for receipt of those samples or they have to enter this same information into a computer which is later uploaded to a server. In addition to being inaccurate and time consuming, these methods only track the delivery of drug samples to medical centers. Once the drug samples are delivered, there are no systems or methods currently available that allow the drug company representative to monitor the inventory of the drug samples that he delivered to various physician offices or to gather information on how the drug samples are being utilized. A drug company representative has to call and/or visit each medical center individually to discuss current inventories and drug sample utilization, wasting time for the drug company representative and the physician or other office staff.

Therefore, the current systems and methods of tracking drug samples are ineffective, complicated, inaccurate, and time consuming from the point of view of the medical offices and drug company representatives. Time is wasted in initial meetings between drug company representatives and physicians, tracking the drug samples medical centers have in stock from each particular drug company, tracking the drug samples that are needed or are running low, and tracking the expiration dates of all of the drug samples. In addition, more time is wasted, and mistakes can be made, filling out all of the forms and inventory logs when new drug samples are received. Even more time is wasted, and more mistakes made, verifying the expiration date of the drug samples, filling out the forms to track the inventory, and filling out the information on the patient chart when the physician dispenses a drug sample to a patient. Finally, with current systems and methods there is no way for drug company representatives to check on the current inventories of drug samples at medical centers and no way to track drug sample utilization.

Therefore, a system and method for drug sample inventory and tracking is needed that simplifies the drug sample check-in process for both physicians and drug company representatives, that simplifies the drug sample dispensing process and recording keeping for physicians, and that provides drug company representatives with access to inventory and drug sample utilization tracking information for drug samples.

### BRIEF SUMMARY OF THE INVENTION

The current invention is a system and method for drug sample inventory and tracking which enables medical centers to track the receipt, dispensing, and inventory of drug samples. In addition, the current invention enables drug companies to retrieve various information regarding their drug samples, any possibly additional information such as patient dispensing information and/or competitor drug sample information, from a remote location.

In one embodiment, the current invention is a system for drug sample inventory and tracking which includes: a central server that stores detailed drug sample information, which identifies drug samples, and drug sample tracking information, which provides information on the receipt, dispensing, and inventory of drug samples at medical centers; a medical center computer system that is located at a remote medical center that can retrieve drug sample information, log drug sample tracking information, and keep track of drug sample inventory; and communications between the central server and the medical center computer system to enable the exchange of the drug sample and drug sample tracking information. Using this system, the method for drug sample tracking and inventory would include: connecting the central server and the medical center computer system to a network; storing the drug sample information and the drug sample tracking information on the central server; logging drug sample tracking information and drug sample inventory information through the medical center computer system; and communicating the drug sample information, drug sample tracking information, and drug sample inventory information between the central server and the medical center computer system. Therefore, the need to fill out paper forms when drug samples are delivered or dispensed has been eliminated thereby saving time and avoiding possible inaccuracies or omissions that occur when filling out papers forms and drug sample inventories are automatically tracked without someone having to manually record every time that a drug sample is received or dispensed and upkeep a paper log of the inventories.

In another embodiment, the system also includes: an input device for entering patient data when a drug sample is dispensed to a patient, communications between the medical center computer system and the central server to enable the exchange of patient information, and storage on the central server for the patient information. Using this system, the method would also include: entering patient information through the medical center computer system; communicating the patient information from the medical center computer system to the central server; and storing the patient information on the central server. Therefore, the need to make manual entries in patient charts and to fill out paper forms when drug samples are dispensed is eliminated.

In another embodiment, the current invention also includes: a drug company computer system that is located at a remote drug company facility that can input drug sample information and communications between the central server and the drug company computer system to enable the exchange of the drug sample information, drug sample tracking information, and possibly patient information between the central server and the drug company computer system. Using this system, the method would also include: connecting a drug company computer system to the network; entering drug sample information through the drug company computer system; and communicating the drug sample information, the drug sample tracking information, and possibly patient information between the drug company computer system and the central server. Therefore, drug companies have access to information regarding the delivery, dispensing, and inventory of their drug samples at multiple medical centers without having to call and/or visit individual medical centers to obtain this information. In addition, drug companies may have access to information as to how their drug samples are being used and the results being obtained.

### BRIEF DESCRIPTION OF THE SEVERAL VIEWS OF THE DRAWING

FIG. 1 sets forth a diagram of the preferred embodiment of the basic structure of the central server.

FIG. 2 sets forth a diagram of the preferred embodiment of the basic structure of the medical center computer system.

FIG. 3 sets forth a diagram of the preferred embodiment of the basic structure of the drug company computer system.

FIG. 4 shows a diagram of the preferred embodiment of the basic structure of the complete system for drug sample inventory and tracking.

FIG. 5 sets forth an exemplary drug sample label with barcodes.

FIG. 6 sets forth a logic flow diagram of the preferred embodiment of the overall method for drug sample inventory and tracking.

FIG. 7 sets forth a logic flow diagram of the preferred embodiment of the step for inputting drug sample information.

FIG. 8 sets forth a logic flow diagram of the preferred embodiment of the step for checking in drug samples.

FIG. 9 sets forth a logic flow diagram of the preferred embodiment of the step for dispensing drug samples.

FIG. 10 sets forth a logic flow diagram of the preferred embodiment of the step for accessing drug sample information.

FIG. 11 sets forth a logic flow diagram of the preferred embodiment for periodically connecting the medical center computer system and central server and updating/synchronizing the information.

FIG. 12 sets forth a logic flow diagram of the preferred embodiment for periodically connecting the drug company computer system and central server and updating/synchronizing the information.

FIG. 13 sets forth an exemplary Login screen.

FIG. 14 sets forth an exemplary Drug Company Main Menu screen.

FIG. 15 sets forth an exemplary Drug Sample Information Entry screen.

FIG. 16 sets forth an exemplary Medical Center Main Menu screen.

FIG. 17 sets forth an exemplary Drug Sample Receipt screen.

FIG. 18 sets forth an exemplary Drug Sample Dispensing screen.

### DETAILED DESCRIPTION OF THE INVENTION

The embodiments of the current invention shown in the attached drawings provide a system and method for drug sample inventory and tracking that allow medical centers to electronically inventory their drug samples, to track the dispensing of drug samples, and to automatically enter drug sample dispensing information into patient charts and allow drug companies to monitor the inventory, dispensing, and utilization of their specific drug samples at various medical centers.

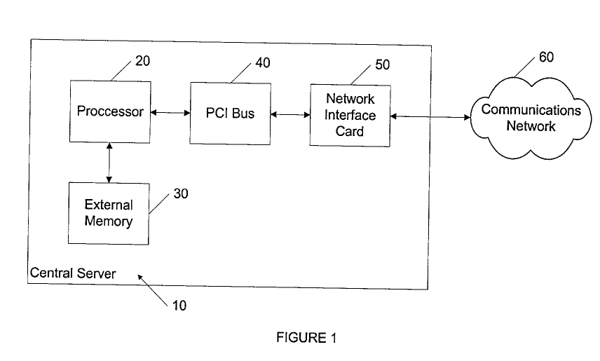

Referring now to the drawings in greater detail, in FIG. 1 there is a central server 10 that is used as a centralized data collection point for various information, such as drug sample information, drug sample tracking information, patient information, and security access information. The central server 10 includes a processor 20, which is a commercially available device designed to operate with an operating system such as Microsoft Corporation Windows, Linux, or other computer operating system. The processor 20 includes internal memory, I/O control to facilitate system integration, integral memory management circuitry for handling all external memory 30, and a PCI bus driver which provides a direct interface with a PCI bus 40.

The PCI bus 40 is an industry standard bus that transfers data between the processor 20 and a number of peripheral controller cards. The PCI bus 40 connects to a communications network 60 through a network interface card 50. The communications network 60 may, for example, be a local area network (“LAN”), a wide area network (“WAN”), or the Internet, and the network interface card 50 provides the required physical and data link layer protocols for that network. In the preferred embodiment, the network interface card 50 links to the Internet via a direct network connection such as such as a T1 line, T3 line, or ISDN line.

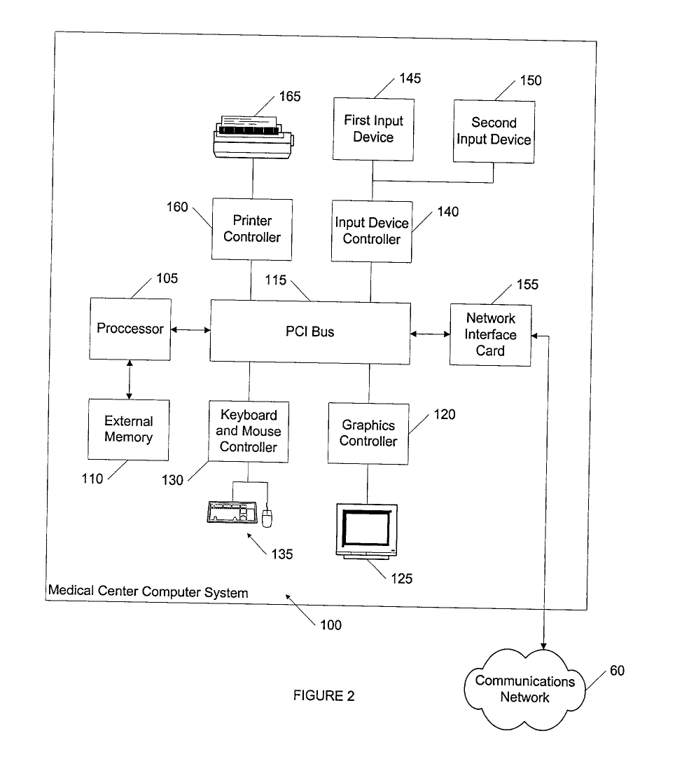

Referring now to FIG. 2, there is a medical center computer system 100 located at a medical center, such as a doctor's office or hospital. Like the central server 10 described above, the medical center computer system 100 includes a processor 105, external memory 110, and a PCI bus driver which provides a direct interface with a PCI bus 115. A graphics controller 120 couples the PCI bus 115 to a CRT monitor 125 through a standard VGA connection, a keyboard and mouse controller 130 receives data that is input through a keyboard and mouse 135, and a printer controller 160 couples the PCI bus 115 to a printer 165 through a standard printer connection. An input device controller 140 also couples the PCI bus 115 to a first input device 145 and a second input device 150.

In the preferred embodiment of the invention the first input device 145 and the second input device 150 are handheld barcode scanners, such as the PSC Model PSRF-1000 wireless bar code scanner sold by PSC Inc., and communicate with the PCI bus 115 via a wireless radio frequency (“RF”) connection. In addition, the first input device 145 and the second input device 150 preferably have the ability to communicate via audio and/or visual means with the user and to receive manual input from the user. The first input device 145 would preferably be located at a reception desk or nurse's station at the medical center and the second input device 150 located where the drug samples are stored in the medical center. It will be understood by one skilled in the art that the first input device 145 and the second input device 150 do not have to be handheld barcode scanners but could be any type of input device such as a magnetic strip reader, optical reader, smart card reader, RFID reader or any other input device that would be capable of inputting information into the system, that they do not have to communicate via a wireless RF connection but could communicate via an infrared connection, a hardwire connection, or any other communications method, and that the system does not require the use of two input devices but can be designed and configured to use as many input devices as desired.

A network interface card 155 connects the medical center computer system 100 to the communications network 60. As will be described in more detail below, the medical center computer system 100 is used to input various information, such as drug sample and patient information, exchange various data with the central server 10, and access data stored on the central server 10.

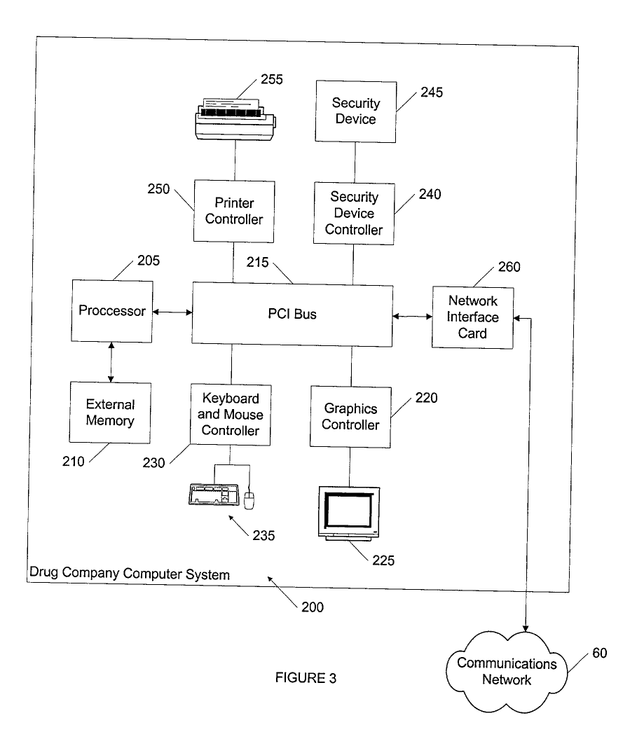

Referring now to FIG. 3, there is a drug company computer system 200 located at a drug company facility. Like the medical center computer system 100 described above, the drug company computer system 200 includes a processor 205, external memory 210, and a PCI bus driver which provides a direct interface with a PCI bus 215. A graphics controller 220 couples the PCI bus 215 to a CRT monitor 225 through a standard VGA connection, a keyboard and mouse controller 230 receives data that is input through a keyboard and mouse 235, and a printer controller 250 couples the PCI bus 215 to a printer 255 through a standard printer connection. A security device controller 240 also couples the PCI bus 215 to a security device 245.

In the preferred embodiment of the invention the security device 245 is a handheld barcode scanner, such as the PSC Model PSRF-1000 wireless bar code scanner sold by PSC Inc., and communicates with the PCI bus 215 via a wireless RF connection. It will be understood by one skilled in the art that the security device 245 does not have to be a handheld barcode scanner but could be any type of security device such as a magnetic strip reader, optical reader, smart card reader, RFID reader or any other security device that would be capable of validating a users authorization to use the system and that it does not have to communicate via a wireless RF connection but could communicate via an infrared connection, a hardwire connection, or any other communications method. Alternatively, if drug company representative authorization is not required or desired at the drug company facility, the security device 245 can be removed from the system.

A network interface card 260 connects the drug company computer system 200 to the communications network 60. As will be described in more detail below, the drug company computer system 200 is used to input various information, such as detailed drug sample information, exchange various data with the central server 10, and access data stored on the central server 10.

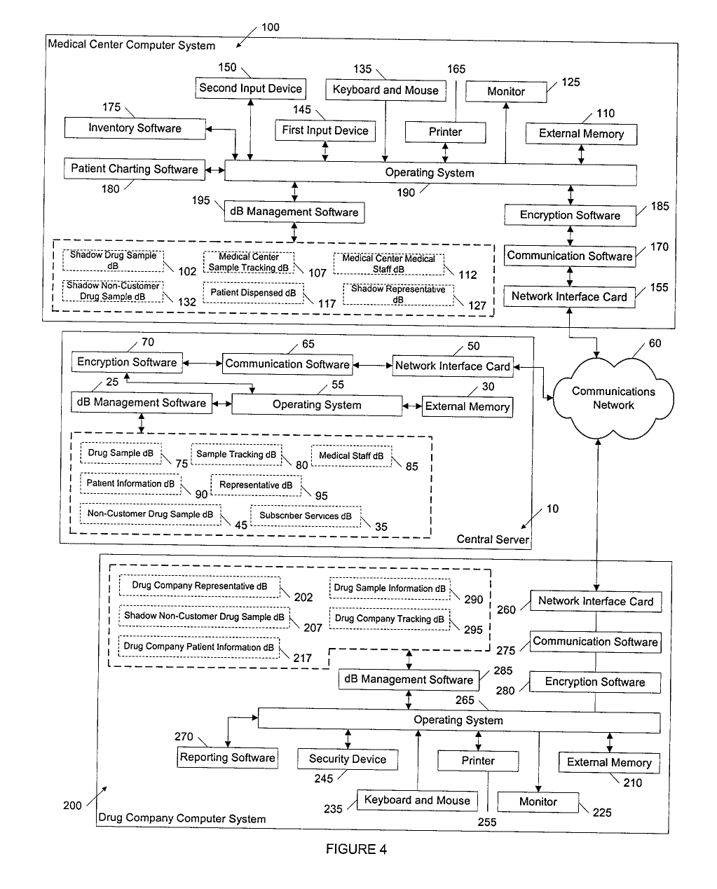

The present invention enables medical centers to automatically record drug sample receipt and dispensing information and gives drug companies remote access to the drug sample receipt and dispensing information and possibly other information such as non-customer drug sample information and non-privileged/non-confidential patient information. Referring particularly to FIG. 4, the central server 10 is connected to the remotely located medical center computer system 100 and the remotely located drug company computer system 200. The systems are connected by a communications network 60 such as a LAN, WAN, or the Internet. A client-server relationship is established between the central server 10 and the medical center computer system 100 and between the central server 10 and the drug company computer system 200 by communication software 65, 170, 275 in the central server 10, medical center computer system 100, and drug company computer system 200 respectively. In the preferred embodiment of the invention, the communication software 170, 275 located on the medical center computer system 100 and the drug company computer system 200 is standard web browser software, such as Internet Explorer by Microsoft Corporation, Netscape Navigator by Netscape, or any other web browser software and the communication software 65 located on the central server 10 may be any communication software with task scheduling capabilities such as Internet Explorer with file upload and download activities identified in the Microsoft Windows Task Scheduler. The communication software 65, 170, 275 operate in combination with their respective operating systems 55, 190, 265 and network interface cards 50, 155, 260 to implement Internet Protocol (“IP”) network connections and Transmission Control Protocol (“TCP”) transport services between the systems 10, 100, and 200. The communications software 65, 170, 275 use a transfer protocol such as Hyper Text Transfer Protocol (“HTTP”) or File Transfer Protocol (“FTP”) to exchange data of various content, including Hypertext Transfer Markup Language (“HTML”) documents, plain text documents, graphic images, Extensible Markup Language (“XML”) documents, etc.

In addition to the communication software 65, the central server 10 also contains a drug sample database 75, a sample tracking database 80, a patient information database 90, a representative database 95, a medical staff database 85, a non-customer drug sample database 45, a subscriber services database 35, database management software 25, and encryption software 70.

The drug sample database 75 contains detailed information on each drug sample provided by the drug companies using the system. As is described in more detail below, the drug sample database 75 is a centralized data collection point for the detailed drug sample information and is periodically updated with data from the drug sample information database 290 located on the drug company computer system 200 and periodically synchronized with the information in the shadow drug sample database 102 located on the medical center computer system 100. An exemplary list of the fields in the drug sample database 75 is set forth in Table I.

TABLE I

| Field | Description |
|---|---|
| SampIdent | Drug Sample Identifier - A unique identifier such as a serial |
| | number that will uniquely identify each separate drug |
| | sample. |
| SampName | The Name of the drug sample. |
| CompName | The name of the drug company that manufactures and/or |
| | distributes the drug sample. |
| ExpDate | The expiration date of the drug sample. |
| Dose | The dosage of the sample. |
| Lot | The lot number of the sample. |
| NDC | The NDC Number of the drug sample. |
| Quantity | The quantity of units included in the drug sample. |

It will be understood by one skilled in the art that the information in the drug sample database 75 could be any information that medical centers and/or drug companies would find useful in identifying drug samples that are delivered, inventoried, and dispensed. In addition, rather than having a single drug sample database 75, there could be multiple databases that organize the detailed drug sample information as desired. Finally, if there is no desire to have detailed drug sample information stored in a centralized location, for example, if the users enter detailed drug sample information each time a drug sample is delivered and/or dispensed, the drug sample database 75 can be removed from the system.

The sample tracking database 80 contains information on the inventory, receipt, and dispensing of each of the drug samples provided by the drug companies using the system. As is described in more detail below, the sample tracking database 80 is a centralized data collection point for drug sample inventory, receipt, and dispensing information and is periodically updated with information from the medical center sample tracking database 107 located on the medical center computer system 100 and periodically updates selected information in the drug company tracking database 295 located on the drug company computer system 200. An exemplary list of the fields in the sample tracking database 80 is set forth in Table II.

TABLE II

| Field | Description |
|---|---|
| SampIdent | Drug Sample Identifier - A unique identifier such as a |
| | serial number that will uniquely identify each separate |
| | drug sample. |
| SampName | The name of the drug sample. |
| CompName | The name of the drug company that manufactures and/or |
| | distributes the drug sample. |
| MedCtrName | The name or numeric identifier of the medical center that |
| | dispenses the drug sample. |
| Dispensing | Detail of the dispensing of each drug sample logged by |
| History | date and time. |
| Inventory | Current inventory of remaining drug samples. Date and |
| | time stamped at each database synchronization. |

It will be understood by one skilled in the art that the information in the sample tracking database 80 could be any information that medical centers and/or drug companies would find useful in tracking the inventory, receipt, and dispensing of drug samples. In addition, rather than having a single sample tracking database 80, there could be multiple databases that organize the sample tracking information as desired.

The patient information database 90 contains select non-confidential/non-privileged information regarding the patients to which drug samples are dispensed. As is described in more detail below, the patient information database 90 is a centralized data collection point for the non-confidential/non-privileged patient information and, if a particular medical center subscribes to provide patient information, is periodically updated with information from the patient dispensed database 117 located on the medical center computer system 100 and, if a particular drug company subscribes to receive patient information, periodically updates selected information for the particular drug company in the drug company patient information database 217 located on the drug company computer system 200. An exemplary list of the fields in the patient information database 90 is set forth in Table III.

TABLE III

| Field | Description |
|---|---|
| PatientID | Patient Identifier - A unique identification number |
| | for each patient to maintain confidentiality. |
| PatientName | Name of the Patient (screened at central server 10). |
| PatientAge | The age of the patient (screened at central server |
| | 10). |
| PatientSex | The gender of the patient (screened at central server |
| | 10). |
| PatientSympt | The patient symptoms. |
| PatientDiag | The patient diagnosis. |
| SampIdent | Drug Sample Identifier - A unique identifier such as |
| | a serial number that will uniquely identify each |
| | separate drug sample. Is linked to the drug sample |
| | database 75. |
| PatientResults | The results of the drug sample dispensed. |
| SampDispPrior | A list of all prior drug samples dispensed. |
| PatientResultsPrior | The results of all prior drug samples dispensed. Is |
| | linked to the particular drug samples listed in the |
| | SampDispPrior field. |
| AdditMeds | Additional medications dispensed or prescribed. |

It will be understood by one skilled in the art that the information in the patient information database 90 could be any information that medical centers and/or drug companies would find useful in tracking, monitoring, and evaluating how certain drug samples are being used. In addition, rather than having a single patient information database 90, there could be multiple databases that organize the information as desired. Finally, if there is no desire or need to track patient data, the patient information database 90 can be removed from the system.

The representative database 95 and medical staff database 85 contain identifying information and system use information for each drug company representative and medical staff member that is authorized to use the system. As is described in more detail below, the representative database 95 and medical staff database 85 are centralized data collection points for the user identification and system use information. The information in the representative database 95 is periodically synchronized with the information in the shadow representative database 127 located on the medical center computer system 100 and selected information relating to a particular drug company in the representative database 95 is periodically synchronized with the information in the drug company representative database 202 located on the drug company computer system 200 for that particular drug company. In addition, the selected information relating to a particular medical center in the medical staff database 85 is periodically synchronized with the information in the medical center medical staff database 112 located on the medical center computer system 100 for that particular medical center. An exemplary list of the fields in the representative database 95 is set forth in Table IV.

TABLE IV

| Field | Description |
|---|---|
| RepID | Representative Identifier - A unique identification number |
| | that will uniquely identify each drug company representa- |
| | tive. These numbers are assigned to each representative |
| | by the drug companies. |
| RepName | The name of the drug company representative. |
| CompName | The name of the drug company that the representative |
| | works for. |
| SampDeliv | The drug samples delivered (listed by the SampID). |
| Delivery | The date and time each drug sample was delivered. |
| QuantDeliv | The quantity of each drug sample delivered. |
| LocDeliv | The medical centers that the drug company representative |
| | has visited. Locations listed by MedCtrName. |

An exemplary list of the fields in the medical staff database 85 is set forth in Table V.

TABLE V

| Field | Description |
|---|---|
| MedStaffID | Medical Staff Identifier - A unique identification number |
| | that will uniquely identify each medical staff member. |
| | These numbers are assigned to each medical staff |
| | member by the medical centers. |
| MedStaffName | The name of the medical staff member. |
| MedStaffAuth | The authorization level of the medical staff member (i.e. |
| | view inventory only, dispense drug samples, etc.) (for |
| | security purposes). |
| MedCtrName | The name or numeric identifier of the medical center that |
| | the medical staff member works for. |
| SampRec | Drug samples received. This field should reconcile with |
| | the drug samples delivered by the drug company |
| | representatives (SampDeliv). |
| SampDisp | The drug samples dispensed by each medical staff |
| | member (listed by SampID). |
| MedStaffLog | System use activity of each medical staff member. |

It will be understood by one skilled in the art that the information in the representative database 95 and medical staff database 85 could be any information that would be useful in verifying that a user is authorized to use the system and to track the system use for that user. In addition, rather than having a single representative database 95 or medical staff database 85, there could be multiple databases for drug company representatives and/or medical staff members that organize the information as desired or a single user database that contains all of the system users. Finally, if the system use of each user is not deemed important or desired, the representative database 95 and/or medical staff database 85 do not have to contain this information and if there is no desire or need to provide system security, the representative database 95 and/or medical staff database 85 can be removed from the system.

The non-customer drug sample database 45 contains detailed information, delivery information, and dispensing information for drug samples from drug companies that do not use the system that are delivered to and dispensed by medical centers using the system. As is described in more detail below, the non-customer drug sample database 45 is a centralized data collection point for the non-customer drug sample information and is periodically updated with information from the shadow non-customer drug sample database 132 located on the medical center computer system 100 and, if a particular drug company subscribes to receive non-customer drug sample information, periodically updates the information in the shadow non-customer drug sample database 207 located on the drug company computer system 200. An exemplary list of the fields in the non-customer drug sample database 45 is set forth in Table VI.

TABLE VI

| Field | Description |
|---|---|
| SampIdentNC | Drug Sample Identifier - A unique identifier such as a |
| | serial number that will uniquely identify each drug |
| | sample. |
| SampNameNC | The name of the drug sample. |
| CompNameNC | The name of the drug company that manufactures and/or |
| | distributes the drug sample. |
| Dose | The dosage of the drug sample. |
| Lot | The lot number of the drug sample. |
| NDC | The NDC number of the drug sample. |
| Quantity | The quantity of units included in the drug sample. |
| RepIDNC | A unique identification number that will uniquely |
| | identify each drug company representative. Since the |
| | representative is from a non-customer drug company, |
| | these numbers are assigned to each representative by the |
| | system software. |
| RepName | The name of the drug company representative. |
| SampDeliv | The drug samples delivered (listed by the SampID). |
| Delivery | The date and time each drug sample was delivered. |
| QuantDeliv | The quantity of each drug sample delivered. |

It will be understood by one skilled in the art that the information in the non-customer drug sample database 207 could be any information that medical centers and/or drug companies would find useful in identifying and/or tracking the inventory, receipt, and dispensing of drug samples from non-customer drug companies that are delivered, inventoried, and dispensed. In addition, rather than having a single non-customer drug sample database 207, there could be multiple databases that organize the non-customer drug sample information as desired. Finally, if there is no desire to have non-customer drug sample information stored in a centralized location, the non-customer drug sample database 207 can be removed from the system.

The subscriber services database 35 contains information on the services/information to be provided to and received from each medical center and drug company using the system. An exemplary list of the fields in the subscriber services database 35 is set forth in Table VII.

TABLE VII

| Field | Description |
|---|---|
| SubID | Subscriber Identifier - A unique identification number that |
| | will uniquely identify each drug company/medical center |
| | using the system. |
| SubLevel | The subscription level for each subscriber (i.e. drug company |
| | or medical center). The subscription level determines the |
| | services/information to be provided to each subscriber (e.g. |
| | MC1, MC2, DC1, DC2, DC3, DC4, DC5). |

In the preferred embodiment of the invention, the subscription levels in the subscriber services database 35 are subscription codes that correspond to certain levels of services/information, for example:

MC1=The medical center will provide drug sample information;

MC2=The medical center will provide drug sample and patient information;

DC1=The drug company receives raw data regarding their drug samples;

DC2=The drug company receives raw data regarding their drug samples and patient information;

DC3=The drug company receives raw data regarding their drug samples and non-customer drug samples;

DC4=The drug company receives raw data regarding their drug samples, non-customer drug samples, and patient information; and

DC5=Used in conjunction with DC1-DC4 above, the drug company receives the data in a customized report format.

It will be understood by one skilled in the art that the information in the subscriber services database 35 does not have to be subscription codes but could be any information that would be useful in determining the services/information to be provided to and/or received from each drug company/medical center. In addition, rather than having a single subcriber services database 35, there could be multiple databases that organize the information as desired. Finally, if there is no service and/or information that is “optional” and each medical center and/or drug company provides and/or receives the same services/information, the subscriber services database 35 can be removed from the system.

The database management software 25 is standard database management software such as Microsoft Access or can be any database management software that can manage the information in the various databases located on the central server 10.

The encryption software 70 is standard data encryption software such as McAfee E-Business Server or can be any encryption software that can encrypt and decrypt data. The encryption software 70 is used to encrypt data that is sent from the central server 10 and to decrypt encrypted data that is received by the central server 10 to protect the data from being intercepted and read by third parties. Alternatively, if it is not desired to protect the information that is sent from and received by the central server 10, the encryption software 70 can be removed from the system.

In addition to the communication software 170, the medical center computer system 100 also contains a shadow drug sample database 102, a medical center sample tracking database 107, a patient dispensed database 117, a medical center medical staff database 112, a shadow representative database 127, a shadow non-customer drug sample database 132, database management software 195, encryption software 185, inventory software 175, and patient charting software 180.

The shadow drug sample database 102 is a duplicate of the drug sample database 75, contains the same fields as the drug sample database 75, and contains detailed information on each drug sample provided by the drug companies using the system. As is described in more detail below, the shadow drug sample database 102 is a local copy of the drug sample database 75 that is used by the medical center computer system 100 and is periodically synchronized with data from the drug sample database 75 on the central server 10. It will be understood by one skilled in the art that, as described for the drug sample database 75 above, the information in the shadow drug sample database 102 could be any information that medical centers and/or drug companies would find useful in identifying drug samples that are delivered, inventoried, and dispensed. In addition, rather than having a single shadow drug sample database 102, there could be multiple databases that organize the detailed drug sample information as desired. Finally, if there is no desire to have detailed drug sample information stored on the medical center computer system 100, for example, if the users enter detailed drug sample information each time a drug sample is delivered and/or dispensed or the system queries the drug sample database 75 on the central server each time a drug sample is delivered and/or dispensed, the shadow drug sample database 102 can be removed from the system.

The medical center sample tracking database 107 is a subset of the sample tracking database 80, contains the same fields as the sample tracking database 80, and contains information on the inventory, receipt, and dispensing of each of the drug samples provided by the drug companies using the system at the particular medical center. As is described in more detail below, the medical center sample tracking database 107 periodically updates the information in the sample tracking database 80 located on the central server 10. It will be understood by one skilled in the art that the information in the medical center sample tracking database 107 could be any information that medical centers and/or drug companies would find useful in tracking the inventory, receipt, and dispensing of drug samples. In addition, rather than having a single medical center sample tracking database 107, there could be multiple databases that organize the drug sample receipt information as desired.

The patient dispensed database 117 is a subset of the patient information database 90, contains the same fields as the patient information database 90, and contains select non-confidential/non-privileged information regarding the patients to which drug samples are dispensed at the particular medical center. As described in more detail below, if a particular medical center subscribes to provide patient information, the patient dispensed database 117 periodically updates the information in the patient information database 90 located on the central server 10. It will be understood by one skilled in the art that, as described for the patient information database 90 above, the information in the patient dispensed database 117 could be any information that medical centers and/or drug companies would find useful in tracking, monitoring, and evaluating how certain drug samples are being used. In addition, rather than having a single patient dispensed database 117, there could be multiple databases that organize the information as desired. Finally, if there is no desire or need to track patient data, the patient dispensed database 117 can be removed from the system.

The medical center medical staff database 112 is a subset of the medical staff database 85, contains the same fields as the medical staff database 85, that contains identifying information and system use information for each medical staff member that is authorized to use the system at the particular medical center. As is described in more detail below, the medical center medical staff database 112 is periodically synchronized with the medical staff member information for the particular medical center from the medical staff database 85 located on the central server 10. It will be understood by one skilled in the art that, as described for the medical staff database 85 above, the information in the shadow medical staff database 112 could be any information that would be useful in verifying that a user is authorized to use the system and to track the system use for that user. In addition, rather than having a single shadow medical staff database 112, there could be multiple databases for medical staff members that organize the information as desired or a single user database that contains all of the system users. Finally, if the system use of each user is not deemed important or desired, the shadow medical staff database 112 does not have to contain this information and if there is no desire or need to provide system security, the shadow medical staff database 112 can be removed from the system.

The shadow representative database 127 is a duplicate of the representative database 95, contains the same fields as the representative database 95, and contain identifying information and system use information for each drug company representative that is authorized to use the system. As is described in more detail below, the shadow representative database 127 is a local copy of the representative database 95 that is used by the medical center computer system 100 and is periodically synchronized with the representative database 95 located on the central server 10. It will be understood by one skilled in the art that, as described for the representative database 95 above, the information in the shadow representative database 127 could be any information that would be useful in verifying that a user is authorized to use the system and to track the system use for that user. In addition, rather than having a single shadow representative database 127, there could be multiple databases for drug company representatives that organize the information as desired or a single user database that contains all of the system users. Finally, if the system use of each user is not deemed important or desired, the shadow representative database 127 does not have to contain this information and if there is no desire or need to provide system security, the shadow representative database 127 can be removed from the system.

The shadow non-customer drug sample database 132 is a duplicate of the non-customer drug sample database 45, contains the same fields as the non-customer drug sample database 45, and contains detailed information, delivery information, and dispensing information for drug samples from drug companies that do not use the system that are delivered to and dispensed by medical centers using the system. As is described in more detail below, the shadow non-customer drug sample database 132 is a local copy of the non-customer drug sample database 45 that is used by the medical center computer system 100 and periodically updates the data in the non-customer drug sample database 45 on the central server 10. It will be understood by one skilled in the art that, as described for the non-customer drug sample database 45 above, the information in the shadow non-customer drug sample database 132 could be any information that medical centers and/or drug companies would find useful in identifying and/or tracking the inventory, receipt, and dispensing of drug samples from non-customer drug companies that are delivered, inventoried, and dispensed. In addition, rather than having a single shadow non-customer drug sample database 132, there could be multiple databases that organize the non-customer drug sample information as desired. Finally, if there is no desire to have non-customer drug sample information stored in a centralized location, the shadow non-customer drug sample database 132 can be removed from the system.

The database management software 195 is standard database management software such as Microsoft Access or can be any database management software that can manage the information in the various databases located on the medical center computer system 100.

The encryption software 185 is standard data encryption software such as McAfee E-Business Server or can be any encryption software that can encrypt and decrypt data. The encryption software 185 is used to encrypt data that is sent from the medical center computer system 100 and to decrypt encrypted data that is received by the medical center computer system 100 to protect the data from being intercepted and read by third parties. Alternatively, if it is not desired to protect the information that is sent from and received by the medical center computer system 100, the encryption software 185 can be removed from the system.

The inventory software 175 is an inventory database using standard database management software such as Microsoft Access, or any database management software that can manage the information concerning the inventory of the various drug samples that must be tracked, or can also be any standard inventory tracking software that is capable of keeping track of the inventory of multiple products and various information regarding the drug samples. The inventory software 175 is used to keep track of the inventory of each drug sample received and dispensed by the medical center and to keep track of various information, such as the expiration date, for each drug sample in inventory at the medical center. Alternatively, if it is not desired to keep track of the inventory electronically, or if the particular medical center does not have the capability of keeping track of the inventory electronically, the inventory software 175 can be removed from the system and the inventory data can be tracked through the information in the medical center sample tracking database 107.

The patient charting software 180 is standard patient charting software such as Alteer Office by Alteer Corporation or can be any patient charting software that keeps electronic patient charts and allows the input of data, such as drug samples dispensed to patients and/or prescriptions written for patients, into the software. The patient charting software 180 is used to keep the charts for each patient of the medical center and more specifically to track the drug samples that are dispensed to each individual patient. Alternatively, if it is not desired to keep electronic patient charts, or if the particular medical center does not have the capability of keeping electronic patient charts but keeps standard paper charts, the patient charting software 180 can be removed from the system.

In addition to the communication software 275, the drug company computer system 200 also contains a drug sample information database 290, a drug company tracking database 295, a drug company representative database 202, a shadow non-customer drug sample database 207, database management software 285, encryption software 280, and reporting software 270.

The drug sample information database 290 is a subset of the drug sample database 75, contains the same fields as the drug sample database 75, and contains detailed information on each drug sample provided by the particular drug company. As is described in more detail below, the drug sample information database 290 periodically updates the information in the drug sample database 75 located on the central server 10. It will be understood by one skilled in the art that, as described above for the drug sample database 75, the information in the drug sample information database 290 could be any information that medical centers and/or drug companies would find useful in identifying drug samples that are delivered, inventoried, and dispensed. In addition, rather than having a single drug sample information database 290, there could be multiple databases that organize the detailed drug sample information as desired. Finally, if there is no desire to have detailed drug sample information, for example, if the users enter detailed drug sample information each time a drug sample is delivered and/or dispensed, the drug sample information database 290 can be removed from the system.

The drug company tracking database 295 is a subset of the sample tracking database 80, contains the same fields as the sample tracking database 80, and contains drug sample receipt, dispensing, and inventory information for the particular drug company's drug samples. The information in the drug company tracking database 295 depends on the information that the particular drug company is interested in and has subscribed to receive. As is described in more detail below, the drug company tracking database 295 is periodically updated with selected information from the sample tracking database 80 located on the central server 10, as appropriate depending on the drug company's subscription. For example, one drug company may want only drug sample inventory information while another drug company may want drug sample inventory, receipt, and dispensing information. It will be understood by one skilled in the art that the information in the drug company tracking database 295 could be any information that drug companies would find useful in tracking the receipt, dispensing, and inventory of drug samples and patient information. In addition, rather than having a single drug company tracking database 295, there could be multiple databases that organize the drug sample tracking information as desired.

The drug company patient information database 217 is a subset of the patient information database 90, contains the same fields as the patient information database 90, and, if a particular drug company subscribes to receive patient information, contains select non-confidential/non-privileged information regarding the patients that are dispensed the drug samples of the particular drug company. The information in the drug company patient information database 217 depends on the patient information that the particular that the particular drug company is interested in and has subscribed to receive. As is described in more detail below, the drug company patient information database 217 is periodically updated with selected information from the patient information database 90 located on the central server 10, as appropriate depending on the drug company's subscription. For example, one drug company may not want any patient information, while another may want to know only the gender and ages of patients that receive their drug samples, and a third may want all of the information regarding patients that receive their drug samples. It will be understood by one skilled in the art that the information in the drug company patient information database 217 could be any information that drug companies would find useful in tracking, monitoring, and evaluating how certain drug samples are being used. In addition, rather than having a single drug company patient information database 217, there could be multiple databases that organize the information as desired. Finally, if there is no desire or need to track patient data, the drug company patient information database 217 can be removed from the system.

The drug company representative database 202 is a subset of the representative database 95, contains the same fields as the representative database 95, and contains identifying information and system use information for each drug company representative at the particular drug company that is authorized to use the system. As is described in more detail below, the drug company representative database 202 is periodically synchronized with the drug representative information for the particular drug company in the representative database 95 located on the central server 10. It will be understood by one skilled in the art that, as described for the representative database 95 above, the information in the drug company representative database 202 could be any information that would be useful in verifying that a user is authorized to use the system and to track the system use for that user. In addition, rather than having a single drug company representative database 202, there could be multiple databases that organize the information as desired. Finally, if the system use of each user is not deemed important or desired, the drug company representative database 202 does not have to contain this information and if there is no desire or need to provide system security, the drug company representative database 202 can be removed from the system.

The shadow non-customer drug sample database 207 is a duplicate of the non-customer drug sample database 45, contains the same fields as the non-customer drug sample database 45, and, if a particular drug company subscribes to receive non-customer drug sample information, contains detailed information, delivery information, and dispensing information for drug samples from drug companies that do not use the system that are delivered to and dispensed by medical centers using the system. The information in the shadow non-customer drug sample database 207 depends on the non-customer drug sample information that the particular drug company has subscribed to receive. As is described in more detail below, the shadow non-customer drug sample database 207 is a duplicate of the non-customer drug sample database 45 that is used by the drug company computer system 200 and is periodically updated with information from the non-customer drug sample database 45 located on the central server 10, as appropriate depending on the drug companies subscription. For example, one drug company may not want any non-customer drug sample information while another drug company may want to know everything that it can about competitors that are delivering drug sample to the same medical centers. It will be understood by one skilled in the art that, as described for the non-customer drug sample database 45 above, the information in the shadow non-customer drug sample database 207 could be any information that drug companies would find useful in identifying and/or tracking the inventory, receipt, and dispensing of drug samples from non-customer drug companies that are delivered, inventoried, and dispensed. In addition, rather than having a single shadow non-customer drug sample database 207, there could be multiple databases that organize the non-customer drug sample information as desired. Finally, if there is no desire to have non-customer drug sample information, the shadow non-customer drug sample database 207 can be removed from the system.

The database management software 285 is standard database management software such as Microsoft Access or can be any database management software that can manage the information in the various databases located on the drug company computer system 200.

The encryption software 280 is standard data encryption software such as McAfee E-Business Server or can be any encryption software that can encrypt and decrypt data. The encryption software 280 is used to encrypt data that is sent from the drug company computer system 200 and to decrypt encrypted data that is received by the drug company computer system 200 to protect the data from being intercepted and read by third parties. Alternatively, if it is not desired to protect the information that is sent from and received by the drug company computer system 200, the encryption software 280 can be removed from the system.

The reporting software 270 is a standard software program, such as Microsoft Access or Microsoft Excel, that can be programmed to query various databases, such as the drug company tracking database 295, and produce various reports based on the information contained in the databases. The reporting software 270 is used by the drug company to produce reports on various information regarding their drug samples and possibly reports on patient information and drug samples from non-customer drug companies depending on what information the drug company has subscribed to receive.

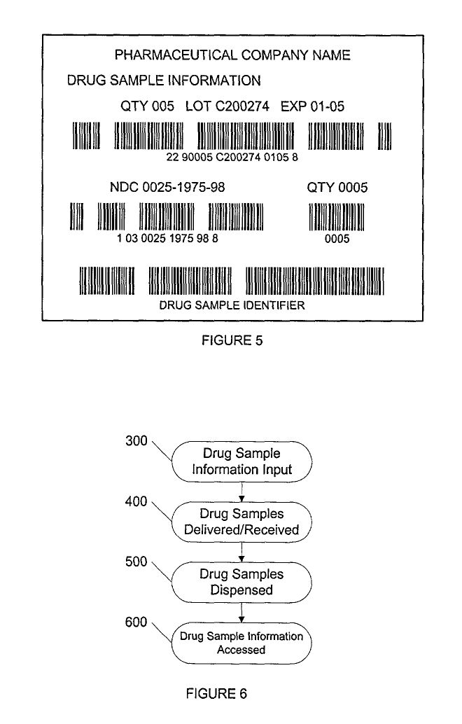

Referring to FIGS. 2 and 4, using the current system a user, such as a drug company representative or medical staff member, can easily and quickly check in a drug sample that is delivered to a medical center by using the keyboard and mouse 135, monitor 125, and first input device 145 to access the system and enter a drug sample identifier for the drug sample being checked in. In the preferred embodiment of the invention, the user would use a handheld barcode scanner to scan a preprinted barcode on the drug sample, such as that shown in FIG. 5, which contains the drug sample identifier. The system will then check the expiration date for the drug sample and if the expiration date has not passed, and the drug sample is from a drug company that uses the system, the information regarding the drug sample and the delivery of the drug sample is automatically saved to the system in the medical center sample tracking database 107 and the inventory program 175, thereby eliminating the need for the drug company representative to fill out any paper forms and for the medical staff member to enter any information in any inventory logs. If the drug sample is from a drug company that does not use the system, the user will be prompted to enter information regarding the drug sample and this information will be automatically saved to the system in the shadow non-customer drug sample database 132.

Similarly, a medical staff member can easily and quickly dispense a drug sample to a patient by using the second input device 150 to access the system and enter a drug sample identifier for the drug sample they wish to dispense. As described above, in the preferred embodiment of the invention, the medical staff member would use a handheld barcode scanner to scan a preprinted barcode on the drug sample. The system checks the expiration date for the drug sample and if the expiration date has not passed, the medical staff member then enters a patient identifier for the patient to which the drug sample is being dispensed. As described above, in the preferred embodiment of the invention, the medical staff member would use a handheld barcode scanner to scan a preprinted barcode on a patient chart which contains the patient identifier. Once the drug sample identifier and patient identifier have been entered, the information regarding the dispensing of that drug sample and the patient to which the drug sample was dispensed is automatically saved to the system in the medical center sample tracking database 107, patient dispensed database 117, inventory software 175, and patient charting software 180, thereby eliminating the need for the medical staff member to fill out any paper forms, make any entries in an inventory log, or make any entry in a patient chart.

Once drug sample receipt, drug sample dispensing, and patient information are being tracked, the medical center computer system 100 will periodically connect to the central server 10 and various databases are updated as follows.

| Medical Center | | |
|---|---|---|
| Computer System | | Central Server |
| (MCCS) Database | Action | (CS) Database |
| shadow drug sample | dB on MCCS and dB on CS | drug sample dB |
| dB 102 | synchronized. | 75 |
| medical center sample | dB on MCCS updates | sample tracking |
| tracking dB 107 | information in dB on CS. | dB 80 |
| medical center medical | dB on MCCS and information | medical staff dB |
| staff dB 112 | related to particular medical | 85 |
| | center in dB on CS | |
| | synchronized. | |
| patient dispensed dB | dB on MCCS updates | patient informa- |
| 117 | information in dB on CS. | tion dB 90 |
| shadow representative | dB on MCCS and dB on CS | representative dB |
| dB 127 | synchronized. | 95 |
| shadow non-customer | dB on MCCS updates | non-customer |
| drug sample dB 132 | information in dB on CS. | drug sample dB |
| | | 45 |

Similarly, the drug company computer system 200 will periodically connect to the central server 10 and various databases are updated as follows.

| | | Drug Company |
|---|---|---|
| Central Server | | Computer System |
| (CS) Database | Action | (DCCS) Database |
| drug sample dB | dB on DCCS updates infor- | drug sample informa- |
| 75 | mation in dB on CS. | tion dB 290 |
| sample tracking | dB on CS updates information | drug company tracking |
| dB 80 | related to the particular drug | dB 295 |
| | company in dB on DCCS. | |
| patient informa- | If subscribed, dB on CS up- | drug company patient |
| tion dB 90 | dates information related to | information dB 217 |
| | the particular drug company | |
| | in dB on DCCS. | |
| representative dB | dB on DCCS and information | drug company repre- |
| 95 | related to particular drug | sentative dB 202 |
| | company in dB on CS | |
| | synchronized. | |
| non-customer | If subscribed, dB on CS up- | shadow non-customer |
| drug sample dB | dates information in dB on | drug sample dB 207 |
| 45 | DCCS. | |

With multiple medical centers and drug companies using the system, this creates a centralized location that can be accessed by drug companies to retrieve consolidated information regarding the receipt, dispensing, and inventory of their drug samples.

Referring to FIGS. 3 and 4, a user at a drug company can access drug sample and/or patient information, depending on what information the drug company has subscribed to receive, by using the keyboard and mouse 235, monitor 225, and security device 245 to access the system and run the reporting software 270. Through the reporting software 270, the user would be able to access the information in various databases such as the drug company tracking database 295, drug company patient information database 217, and shadow non-customer drug sample database 207 and create reports based on this information, thereby eliminating the need for the drug company representatives to take time to call and/or visit individual medical centers to retrieve drug sample inventory information and giving the drug company timely information regarding the receipt, delivery, and inventory of their drug sample, and possibly patient information and competitors drug samples, at multiple medical centers.

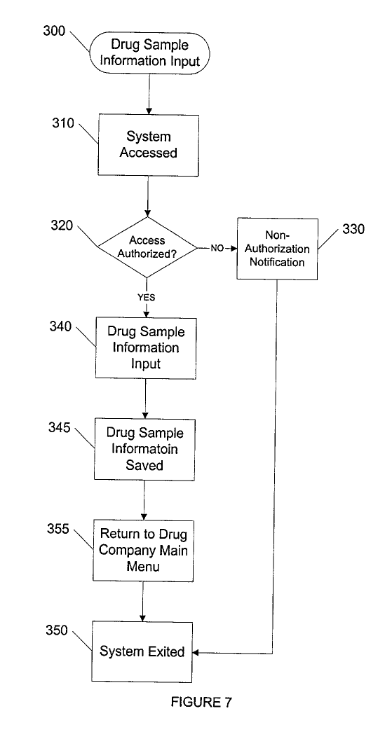

In FIG. 6 there is illustrated the preferred embodiment of the overall method for drug sample inventory and tracking using the system described above. The overall method shown in FIG. 6 shows the major steps that are taken for a single drug sample from the time that a drug company has the drug sample ready for distribution through the time that the drug sample is dispensed from a medical center and the receipt, dispensing, and inventory information is accessed. The details of each of the major steps are illustrated in FIGS. 7-10 and are described in detail below. While these steps are shown in a specific order, in practice these steps may be performed for multiple drug samples, multiple drug companies, and multiple medical centers, in various order at any given time. For example, a medical center may be checking in a drug sample that it has received at the same that a drug company is entering drug sample information for a new drug sample that it will be distributing.

At process block 300, once a drug company has a particular drug sample ready for distribution, detailed drug sample information, is input into the drug company computer system 200 and saved in the drug sample information database 290. This detailed drug sample information is used to fully identify drug samples that are being tracked through the system. The details of this step are shown in FIG. 7 and are described in greater detail below. Alternatively, if there is no desire or need to identify the details of a drug sample through the system, for example if the detailed information regarding a drug sample is manually input when a drug sample is delivered or dispensed, then this step can be skipped.

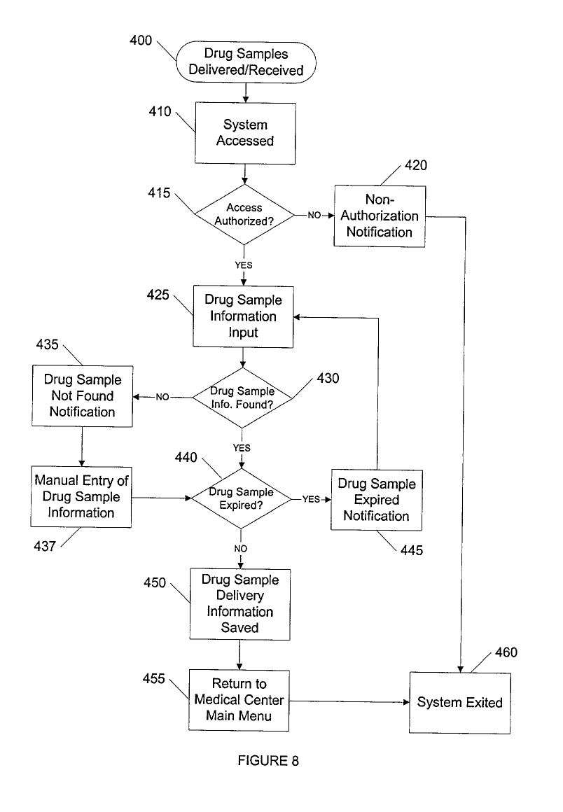

At process block 400, when a drug company representative delivers a drug sample to a medical center, such as a doctor's office or hospital, information regarding the delivery of the drug sample is input into the medical center computer system 200. If the drug sample is from a drug company that uses the system, the information is saved in the medical center sample tracking database 107 and inventory software 175 and the drug sample is stocked at the medical center. If the drug sample is from a drug company that does not use the system, the information is saved in the shadow non-customer drug sample database 132 and inventory software 175 and the drug sample is stocked at the medical center. The details of this step are shown in FIG. 8 and are described in greater detail below.

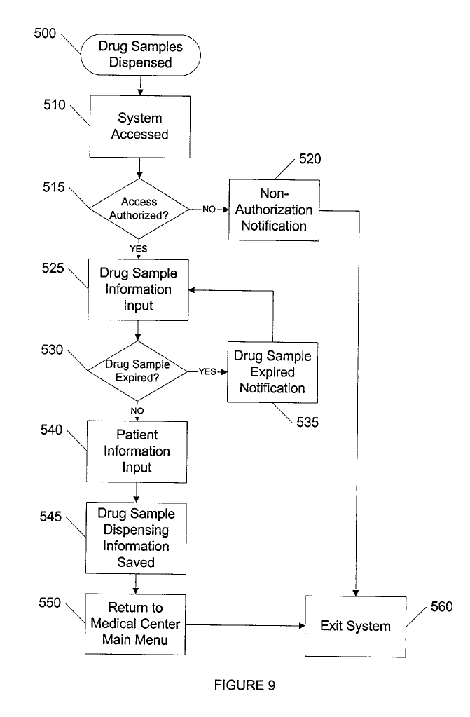

At process block 500, a drug sample is dispensed to a patient from the medical center and information regarding the dispensing of the drug sample and the patient the drug sample was dispensed to is input into the medical center computer system 100 and saved in the medical center sample tracking database 107, patient dispensed database 117, inventory software 175, patient charting software 180, and possibly the shadow non-customer drug sample database 132. The details of this step are shown in FIG. 9 and are described in greater detail below.

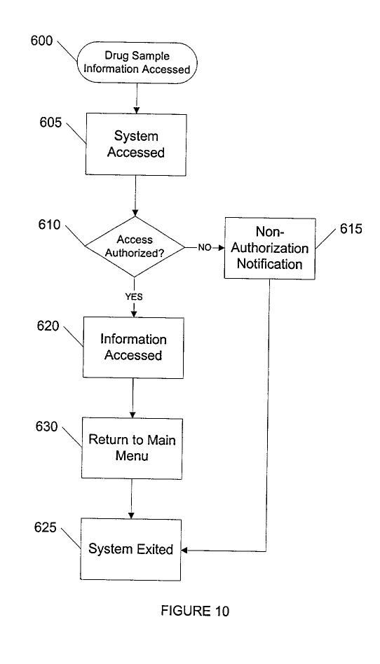

At process block 600, drug sample and/or patient information is accessed. The details of this step are shown in FIG. 10 and are described in greater detail below.

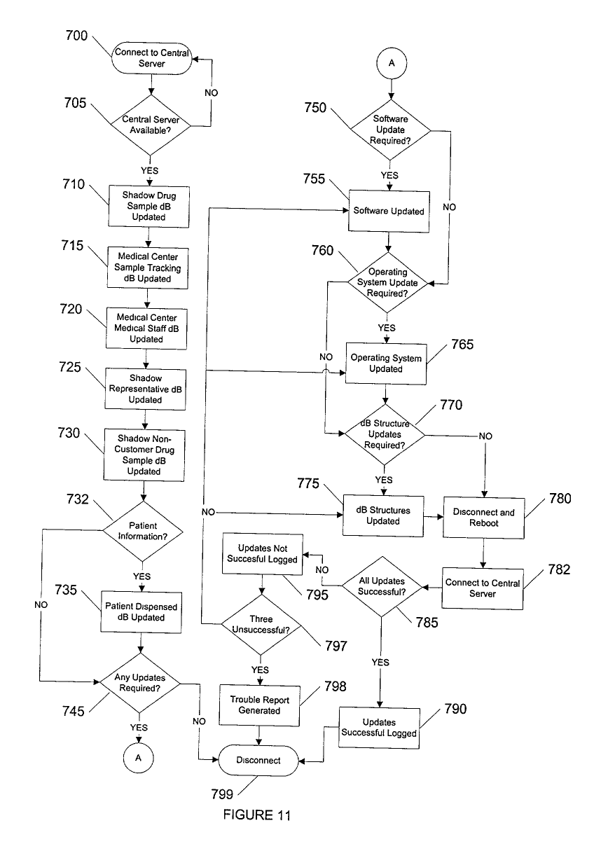

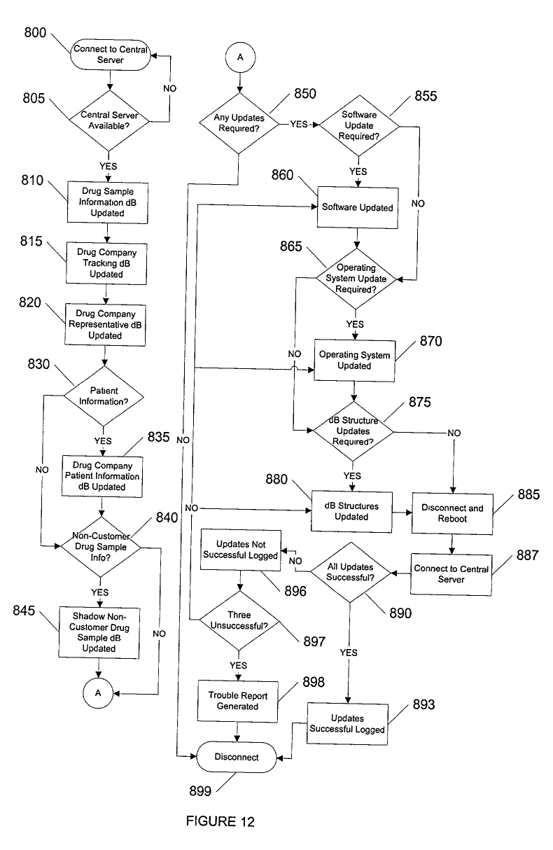

As described above, while all of the above-referenced steps are occurring, the medical center computer system 100 and the drug company computer system 200 are periodically connecting to the central server 10 and various databases are updated/synchronized and various “housekeeping” functions are performed. The details of these steps are shown in FIGS. 11 and 12 and are described in greater detail below.

Referring now to FIG. 7, a user at a drug company, such as a drug company representative, can enter detailed drug sample information by following a series of steps.

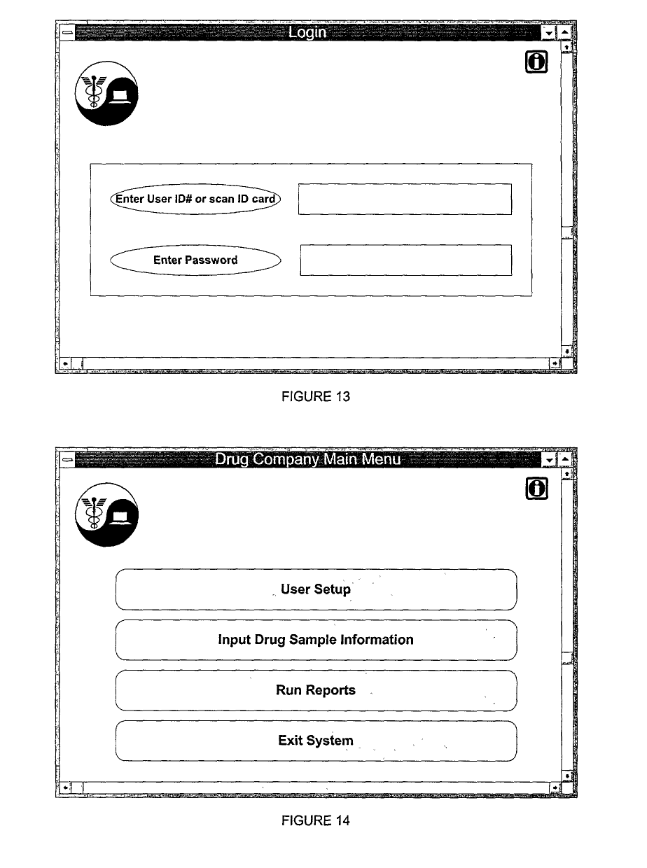

The first step, as shown at process block 310, is to access the drug company computer system 200. A Login Screen, such as that shown in FIG. 13, is displayed and a user, such as a drug company representative, inputs a representative identifier using the security device 245. The representative identifier could be any unique identifier such as the user's name, the user's social security number, an identification number, or any other unique identification desired. As described above, in the preferred embodiment of the invention the security device 245 is a handheld barcode scanner. The user would use the handheld barcode scanner to scan a preprinted barcode, such as from an identification card, that contains the representative identifier. In addition, as an added measure of security, the user could also be required to input a password, for example by using the security device 245 or the keyboard and mouse 235, before access to the system would be granted. It will be understood by one skilled in the art that many different types of security devices may be used and the method in which the representative identifier is entered depends on the type of security device used. For example, if the security device 245 is a magnetic strip reader, the representative identifier would be recorded on a magnetic strip, such as on an identification card, and the user would swipe the identification card through the magnetic strip reader.

Once the representative identifier has been entered, it is determined if the user is authorized to access the system, as shown in process block 320. The representative identifier is checked against the RepID field in the drug company representative database 202. If the representative identifier is not found in the drug company representative database 202, the user is notified that they are not authorized to use the system (process block 330) through feedback through the security device 245, if the security device has feedback capability, or through the monitor 225, and the system is exited (process block 350). If the representative identifier is found in the drug company representative database 202, a Drug Company Main Menu, such as that shown in FIG. 14, is displayed through the monitor 225.

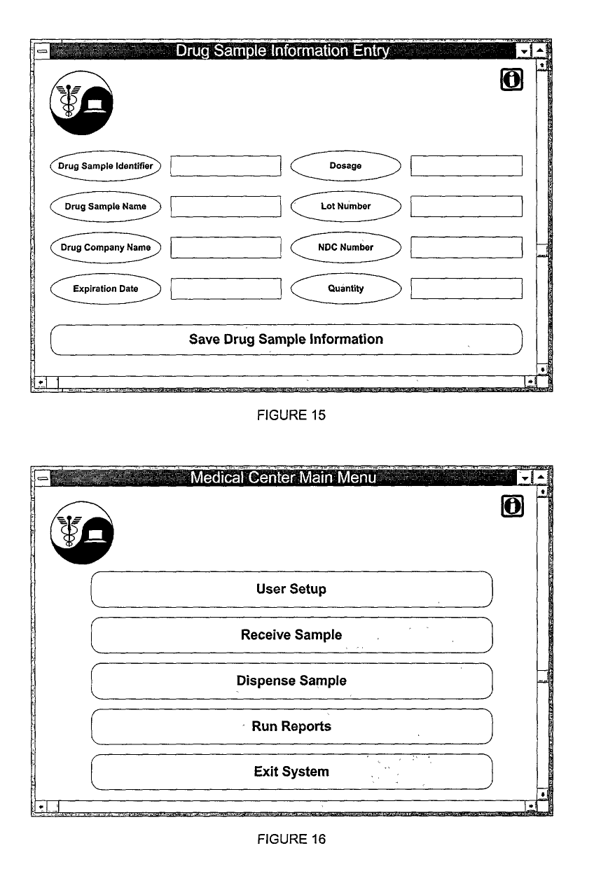

Once the user has logged onto the system, the user then inputs the detailed drug sample information, as shown in process block 340. From the Drug Company Main Menu, the user selects the Input Drug Sample Information option and a Drug Sample Information Entry screen, such as that shown in FIG. 15, is displayed. Using the keyboard and mouse 235, the user then enters the detailed drug sample information requested. Once the drug sample information has been entered, the user selects Save Drug Sample Information, the detailed drug sample information is saved in the drug sample information database 290, as shown in process block 345, and the user is returned to the Drug Company Main Menu, as shown in process block 355.

When the user has entered the drug sample information for all of the drug samples, the user selects Exit System from the Drug Company Main Menu and exits the system, as shown in process block at 350. When the user exits the system, various information regarding their use of the system, such as the date the system was accessed, the drug sample information entered, etc., is saved to the drug company representative database 202.

Referring now to FIG. 8, a drug sample is delivered to a medical center and checked in by a user, such as a medical staff member or drug company representative, by following a series of steps.

The first step, as shown in process block 410, is for a user, such as a medical staff member, to access the medical center computer system 100. A Login Screen, such as that shown in FIG. 13, is displayed through the monitor 125, and the user inputs a medical staff identifier using the first input device 145. The medical staff identifier could be any unique identifier such as the user's name, the user's social security number, an identification number, or any other unique identification desired. As described above, in the preferred embodiment of the invention the first input device 130 is a handheld barcode scanner. The users uses the handheld barcode scanner to scan a preprinted barcode, such as from an identification card, that contains the medical staff identifier. In addition, as an added measure of security, the user could also be required to input a password, for example by using the first input device 145 or the keyboard and mouse 135, before access to the system would be granted. It will be understood by one skilled in the art that the first input device 130 could also be any other type of input device and the method in which the medical staff identifier is entered depends on the type of input device used.

Once the medical staff identifier has been entered, it is determined if the user is authorized to access the system, as shown in process block 415. The medical staff identifier is checked against the MedStaffID field in the medical center medical staff database 112. If the medical staff identifier is not found in the medical center medical staff database 112, the user is notified that they are not authorized to use the system (process block 420) through feedback through the first input device 145, if the first input device has feedback capability, or through the monitor 125, and the system is exited (process block 460). If the medical staff identifier is found in the medical center medical staff database 112, a Medical Center Main Menu, such as that shown in FIG. 16, is displayed.

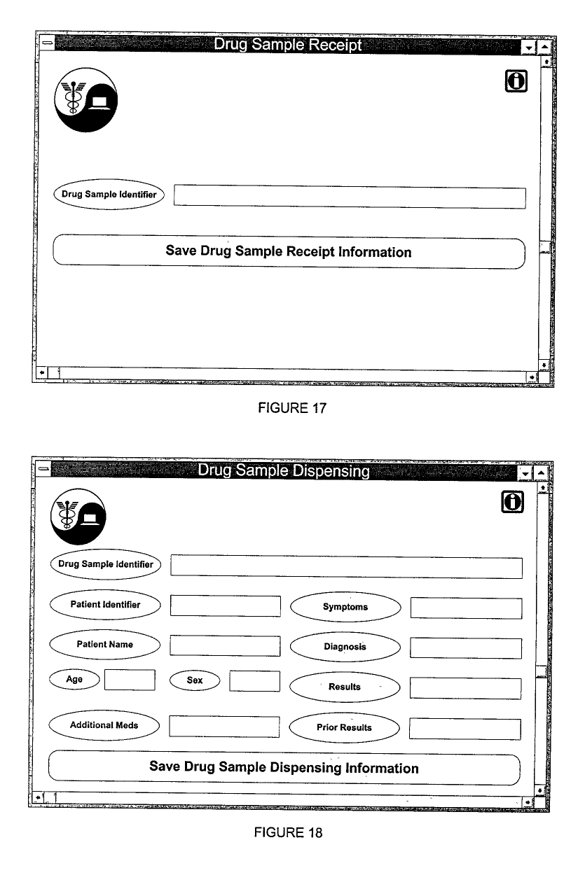

Once the user has logged onto the system, the user then inputs information regarding the drug sample received, as shown in process block 425. From the Medical Center Main Menu, the user selects Receive Sample, a Drug Sample Receipt screen, such as that shown in FIG. 17, is displayed, and the user inputs a drug sample identifier using the first input device 145. The drug sample identifier could be any unique identifier such as a serial number for the drug sample. As described above, in the preferred embodiment of the invention the first input device 145 is a handheld barcode scanner. The user uses the handheld barcode scanner to scan a preprinted barcode, such as that shown in FIG. 5 (preferably printed on the drug sample packaging), that contains the drug sample identifier. It will be understood by one skilled in the art that the first input device 145 could also be any other type of input device and the method in which the drug sample identifier is entered depends on the type of input device used.

Once the drug sample identifier has been entered, the user selects Save Drug Sample Receipt Information and it is determined if the detailed drug sample information for that drug sample has been entered into the system, as shown in process block 430. The drug sample identifier is checked against the SampIdent field in the shadow drug sample database 102. If the drug sample identifier is not found in the shadow drug sample database 102, the user is notified that the drug sample identifier could not be found (process block 435) by feedback through the first input device 145, if the first input device has feedback capability, or through the monitor 125, and a Drug Sample Information Entry Screen, such as that shown in FIG. 15, is displayed. At the Drug Sample Information Entry Screen, the user inputs detailed information regarding the drug sample received, as shown in process block 437, and selects Save Drug Sample Information. If the drug company supplying the drug sample is a user of the system, the detailed drug sample information is saved in the shadow drug sample database 102. If the drug company supplying the drug sample is not a user of the system, the detailed drug sample information is saved in the shadow non-customer drug sample database 132. If the drug sample identifier is found in the shadow drug sample database 102, the detailed drug sample information is retrieved from the shadow drug sample database 102.

Once the detailed drug sample information has been retrieved, either through manual entry or retrieved from the shadow drug sample database 102, the expiration date of the drug sample is checked, as shown in process block 440. The expiration date for the drug sample is checked against the current date, which can be obtained from an internal clock or by any other method of obtaining the current date. If the expiration date for the drug sample has passed, the user is notified that the drug sample has expired (process block 445) by feedback through the first input device 145, if the first input device has feedback capability, or through the monitor 125, and the user is returned to the Drug Sample Receipt screen. If the expiration date for the drug sample has not passed, the drug sample delivery information is saved as described below.

Once the detailed drug sample information has been entered, and the expiration date verified, the drug sample delivery information is saved, as shown in process block 450. If the drug sample is from a drug company that uses the system, various information regarding the drug sample delivery, such as the detailed drug sample information, the date delivered, the quantity delivered, etc., is saved in the medical center sample tracking database 107 and in the inventory software 175. If the drug sample is from a drug company that does not use the system, various information regarding the drug sample delivery is saved in the shadow non-customer drug sample database 132 and in the inventory software 175.

Once the drug sample delivery information has been saved, the user is returned to the Medical Center Main Menu, as shown in process block 455. When the user has checked in all of the drug samples received, the user selects Exit System from the Medical Center Main Menu and exits the system (process block at 460). When the user exits the system, various information regarding their use of the system, such as the date the system was accessed, the drug samples checked in, etc., is saved to the medical center medical staff database 112.

Referring now to FIG. 9, a drug sample is dispensed to a patient by a user, such as a medical staff member, by following a series of steps.

The first step, as shown in process block 510, is for the user to access the medical center computer system 100. Similar to the process described above, a Login Screen, such as that shown in FIG. 13, is displayed and the user inputs a medical staff identifier using the second input device 150. As described above, in the preferred embodiment of the invention the second input device 150 is a handheld barcode scanner and the user would use the handheld barcode scanner to scan in a preprinted barcode that contains the medical staff identifier. In addition, as an added measure of security, the user could also be required to input a password before access to the system would be granted. It will be understood by one skilled in the art that the second input device 150 could also be any other type of input device and the method in which the medical staff identifier is entered depends on the type of input device used. In addition, it is understood that the system can be designed to use any number of input devices as desired.

Once the medical staff identifier has been entered, it is determined if the user is authorized to access the system, as shown in process block 515. The medical staff identifier is checked against the MedStaffID field in the medical center medical staff database 112. If the medical staff identifier is not found in the medical center medical staff database 112, the user is notified that they are not authorized to use the system (process block 520) by feedback through the second input device 150, if the second input device has feedback capability, or through the monitor 125, and the system is exited (process block 560). If the medical staff identifier is found in the medical center medical staff database 112, a Medical Center Main Menu, such as that shown in FIG. 16, is displayed.

Once the user has logged onto the system, the user inputs information regarding the drug sample being dispensed, as shown in process block 525. From the Medical Center Main Menu, the user selects Dispense Sample, a Drug Sample Dispensing screen, such as that shown in FIG. 18, is displayed, and the user inputs the drug sample identifier for the drug sample being dispensed using the second input device 150. As stated above, in the preferred embodiment the second input device 150 is a handheld barcode scanner and the user uses the handheld barcode scanner to scan a preprinted barcode that contains the drug sample identifier. The drug sample identifier is checked against the SampIdent field in the shadow drug sample database 102 and the detailed drug sample information is retrieved from the shadow drug sample database 102.

Once the detailed drug sample information has been retrieved, the expiration date of the drug sample is checked, as shown in process block 530. The expiration date for the drug sample, which is part of the detailed drug sample information, is checked against the current date, which can be obtained from an internal clock or by any other method of obtaining the current date. If the expiration date for the drug sample has passed, the user is notified that the drug sample has expired (process block 535) by feedback through the second input device 150 and/or monitor 125, and the information that the drug sample is expired, and discarded, is saved in the medical center sample tracking database 107, or shadow non-customer drug sample database 132, and in the inventory software 175, and the user is returned to process block 525 to input a new drug sample identifier. If the expiration date for the drug sample has not passed, the user inputs patient information for the patient to which the drug sample is being dispensed, as shown in process block 540. After entering the drug sample identifier, the user continues on the Drug Sample Dispensing screen and inputs a patient identifier using the second input device 150. In the preferred embodiment of the invention, the second input device 150 is a handheld barcode scanner and the user uses the handheld barcode scanner to scan a preprinted barcode, such as from a patient chart, that contains the patient identifier. Basic patient information, such as the patient name, age, and gender is retrieved from the patient charting software 180 based on the patient identifier. If the particular system is not using patient charting software 180, the user would then use the keyboard and mouse 135 to enter the basic patient information. The user then uses the keyboard and mouse 135 to enter additional information, for example whether the patient is part of a clinical trial, the patients symptoms, etc., and selects Save Drug Sample Dispensing Information.

Once the drug sample identifier, patient identifier, and additional patient information have been entered, the drug sample dispensing information is saved, as shown in process block 545. Information regarding the drug sample dispensed is saved to the system. If the drug sample is from a drug company that uses the system, various information regarding the dispensing of the drug sample, such as the date dispensed, the quantity dispensed, etc., is saved in the medical center sample tracking database 107 and in the inventory software 175. If the drug sample is from a drug company that does not use the system, various information regarding the dispensing of the drug sample is saved in the shadow non-customer drug sample database 132 and in the inventory software 175. Various information regarding the patient to which the drug sample was dispensed, such as the patient's name, age, gender, symptoms, additional medications, etc., is saved in the patient dispensed database 117 and information regarding the dispensing of the sample to the patient, such as the date, the drug sample dispensed, the quantity, etc., is saved in the patient charting software 180. Alternatively, if the system does not use the patient charting software 180, the information regarding the dispensing of the sample to the patient could be printed using the printer 165 and a hardcopy placed in the patient chart.

When the drug sample dispensing information has been saved, the user is returned to the Medical Center Main Menu, as shown in process block 550, where the user can choose to dispense additional drug samples. After the user has entered the drug sample dispensing information for all of the drug samples that are being dispensed, the user selects Exit System and exits the system, as shown at process block at 560. When the user exits the system, various information regarding the user's use of the system, such as the date the system was accessed, the drug samples received and/or dispensed by the user, etc., is saved to the medical center medical staff database 112.

Referring now to FIG. 10, drug sample information, and possibly patient information, is accessed following a series of steps.

The first step, as shown in process block 605, is for the user to access the system. As described above, if the user is a medical staff member accessing the medical center computer system 100, a Login Screen, such as that shown in FIG. 13, is displayed and the user uses the first inputs device 145, or possibly the keyboard and mouse 135, to input a medical staff identifier. It is then determined if the user is authorized to access the system, as shown in process block 610 by checking the medical staff identifier against the MedStaffID field in the medical center medical staff database 112. If the medical staff identifier is not found in the medical center medical staff database 112, the user is notified that they are not authorized to use the system (process block 615) and the system is exited (process block 625). If the medical staff identifier is found in the medical center medical staff database 112, a Medical Center Main Menu, such as that shown in FIG. 16, is displayed.

Similarly, if the user is a drug representative accessing the drug company computer system 200, a Login Screen, such as that shown in FIG. 13, is displayed and the user uses the security device 245, or possibly the keyboard and mouse 235, to input a representative identifier. It is then determine if the user is authorized to access the system (process block 610) by checking the representative identifier against the RepID field in the drug company representative database 202. If the representative identifier is not found in the drug company representative database 202, the user is notified that they are not authorized to use the system (process block 615) and the system is exited (process block 625). If the representative identifier is found in the drug company representative database 202, a Drug Company Main Menu, such as that shown in FIG. 14, is displayed.

Once the system has been accessed, the user can then access various drug sample and patient information. If a medical staff member has accessed the medical center computer system 100, the medical staff member could choose Run Reports from the Medical Center Main Menu and: (1) run the inventory software 175 to view, update, or print information regarding the inventory of drug samples at the medical center; (2) run the patient charting software 180 to view, update, or print patient information in the patient charts; or (3) use the database management software 195 to view or print drug sample inventory and tracking information from the medical center sample tracking database 107 or patient drug sample dispensing information from the patient dispensed database 117.

If a drug company representative has accessed the drug company computer system 200, the drug company representative could choose the Run Reports from the Drug Company Main Menu and: (1) run the reporting software 270 to view, create, or print reports regarding their drug samples, patient information for patients that have been dispensed the company's drug samples, and/or non-customer drug sample drug sample information, depending on the information the drug company has subscribed to receive; or (2) use the database management software 285 to view or print the raw information from the drug company tracking database 295, drug company patient information database 217, and/or shadow non-customer drug sample database 207, depending on the information the drug company has subscribed to receive. Alternatively, rather than providing a drug company raw data regarding their drug samples, information on patient's that have received their drug samples, or non-customer drug sample information, a pre-selected report or other communication can periodically be sent to the drug company via facsimile, email, on CD-ROM, or by allowing access to a website.

When the medical staff member/drug company representative is done running reports, they are returned to the Medical Center Main Menu/Drug Company Main Menu, as shown in process block 630, where they can exit the system, as shown at process block at 625. When the medical staff member/drug company representative exits the system, various information regarding their use of the system, such as the date the system was accessed, the reports run, the information accessed, etc., is saved to the medical center medical staff database 112/drug company representative database 202.

As stated above, the information contained in the databases on the medical center computer system 100 and the drug company computer system 200 must be periodically updated/synchronized with the information in the databases on the central server 10 to create a centralized location for all information. In addition to the updating/synchronizing of the databases, various “housekeeping” functions are also performed.

Referring now to FIG. 11, the medical center computer system 100 connects to the central server 10 and updates/synchronizes various databases and performs various “housekeeping” functions by following a series of steps.

The first step, as shown in process block 700, is for the medical center computer system 100 to attempt to connect to the central server 10 through the communications network 60. If the medical center computer system 100 connects to the central server 10 via the Internet, at a predetermined time the medical center computer system 100 establishes a secured connection to the IP address of the central server 10 using the communications software 170 and the encryption software 185. The medical center computer system 100 can access the Internet using a dial-up connection, cable modem, DSL line, ISDN line, a T1 connection, or a T3 connection. The medical center computer system 100 can also access the central server 10 using a direct dial-up modem connection to the central server 10, such as through a toll-free or other telephone number. The medical center computer system 100 first determines if the central server 10 is available, as shown in process block 705. If the central server 10 is not available, for example if the central server 10 is busy communicating with a maximum number of connections to other computer systems, the medical center computer system 100 attempts to connect again at a predetermined time interval. If the central server 10 is available, the medical center computer system 100 completes the connection to the central server 10.

Once the medical center computer system 100 is connected to the central server 10: (1) the information in the shadow drug sample database 102 is synchronized with the information in the drug sample database 75 (process block 710); (2) the information in the medical center sample tracking database 107 updates the information in the sample tracking database 80 (process block 715); (3) the information in the medical center medical staff database 112 is synchronized with the information in the medical staff database 85 relating to the particular medical center (process block 720); (4) the information in the shadow representative database 127 is synchronized with the information in the representative database 95 (process block 725); and (5) the information in the shadow non-customer drug sample database 132 updates the information in the non-customer drug sample database 45 (process block 730). The central server 10 then determines, by checking the subscriber services database 35, if the medical center has subscribed to provide patient information, as shown in process block 732. If the medical center has subscribed to provide patient information, the information in the patient dispensed database 117 updates the information in the patient information database 90, as shown in process block 735. All of the information transferred between the medical center computer system 100 and the central server 10 is encrypted by the encryption software 185, 70 before it is transmitted over the communications network 60 and decrypted using the encryption software 185, 70 once received.

Once all of the relevant databases have been updated/synchronized, the central server 10 queries the medical center computer system 100 to see if any updates are required, as shown in process block 745. To determine if any upgrades are required, the central server 10 queries a ‘versions’ file located on the medical center computer system 100 and compares it against its own ‘versions’ file, which tracks the current versions of all software applications, operating systems, and database structures. The central server 10 could assign a unique version ID for each application and file within the ‘versions’ file for this purpose. Alternatively, using Microsoft Windows, the versions of the software applications, operating systems, and database structures located on the medical center computer system 100 could be identified by the “Properties” attribute of the application or file or the “Last Modified Date” of each application or file. If there are no updates required, the medical center computer system 100 disconnects from the central server 10 (process block 799). If there are updates required, the central server 10 determines what updates are required and uploads the required updates to the medical center computer system 100, which then installs the updates.

If the central server 10 determines that updates are required, it first queries the medical center computer system 100 to determine what software application(s), if any, need to be updated, as shown in process block 750. If there are software updates required, they are uploaded to the medical center computer system 100 and installed, as shown in process block 755. Once the software updates have been uploaded and installed, or if there are no software updates required, the central server 10 queries the medical center computer system 100 to determine what operating system updates, if any, are required, as shown in process block 760. If there are operating system updates required, they are uploaded to the medical center computer system 100 and installed, as shown in process block 765. Once the operating system updates have been uploaded and installed, or if there are no operating system updates required, the central server 10 queries the medical center computer system 100 to determine what database structure(s), if any, need to be updated, as shown in process block 770. If there are database structure updates required, they are uploaded to the medical center computer system 100 and installed, as shown in process block 775. Once the database structure updates have been uploaded and installed, or if there are no database structure updates required, the medical center computer system 100 disconnects from the central server 10 and reboots, as shown in process block 780. All of the updates transmitted from the central server 10 to the medical center computer system 100 are encrypted by the encryption software 70, transmitted over the communications network 60, and decryption using the encryption software 185 once received.

Once the medical center computer system 100 completes its reboot, it again connects to the central server 10 through the communications network 60 as described above, as shown in process block 782. Once the medical center computer system 100 has reconnected to the central server 10, the central server 10 queries the medical center computer system 100, as described above, to verify that all of the updates were successful, as shown in process block 785. If all of the updates were successful, the central server 10 logs that all updates were successful, as shown in process block 790, and disconnects (process block 799). If all of the updates were not successful, the central server 10 logs which updates were not successful, as shown in process block 795, and checks if there have been three unsuccessful updates, as shown in process block 797. If there have not been three unsuccessful updates, the update procedure for the unsuccessful updates is repeated by returning to process block 755, 765, or 775, as required. If there have been three unsuccessful updates, or if the medical center computer system 100 fails to reboot and reconnect to the central server 10, the central server 10 generates a trouble report, as shown in process block 798, which assigns a technician to be dispatched to the medical center for further diagnostics and/or repair.

Referring now to FIG. 12, the drug company computer system 200 connects to the central server 10 and updates/synchronizes various databases and performs various “housekeeping” functions by following a series of steps.

The first step, as shown in process block 800, is for the drug company computer system 200 to attempt to connect to the central server 10 through the communications network 60. If the drug company computer system 200 connects to the central server 10 via the Internet, at a predetermined time the drug company computer system 200 establishes a secured connection to the IP address of the central server 10 using the communications software 275 and encryption software 280. The drug company computer system 200 can access the Internet using a dial-up connection, cable modem, DSL line, ISDN line, a T1 connection, or a T3 connection. The drug company computer system 200 can also access the central server 10 using a direct dial-up modem connection to the central server 10, such as through a toll-free or other telephone number. The drug company computer system 200 first determines if the central server 10 is available, as shown in process block 805. If the central server 10 is not available, for example if the central server is busy communicating with a maximum number of connections to other computer systems, the drug company computer system 200 attempts to connect again at a predetermined time interval. If the central server 10 is available, the drug company computer system 200 completes the connection to the central server 10.

Once the drug company computer system 200 is connected to the central server 10: (1) the information in the drug sample information database 290 updates the information in the drug sample database 75 (process block 810); (2) the information in the drug company tracking database 295 is updated with the information relating to the particular drug company in the sample tracking database 80 (process block 815); and (3) the information in the drug company representative database 202 is synchronized with the information relating to the particular drug company in the representative database 95 (process block 820).

The central server 10 then determines, by checking the subscriber services database 35, if the particular drug company has subscribed to receive patient information, as shown in process block 830. If the drug company has subscribed to receive patient information, the information in the drug company patient information database 217 is updated with the information relating to the particular drug company in the patient information database 90 (process block 835). Once the drug company patient information database 217 has been updated, or if the drug company has not subscribed to receive patient information, the central server 10 determines, by checking the subscriber services databases 35, if the particular drug company has subscribed to received non-customer drug sample information, as shown in process block 840. If the drug company has subscribed to receive non-customer drug sample information, the information in the shadow non-customer drug sample database 207 is updated with the information in the non-customer drug sample database 45 (process block 845). All of the information transferred between the drug company computer system 200 and the central server 10 is encrypted by the encryption software 280, 70 before it is transmitted over the communications network 60 and decrypted using the encryption software 280, 70 once received.

Once all of the relevant databases have been updated/synchronized, the central server 10 queries the drug company computer system 200 to see if any upgrades are required, as shown in process block 850. To determine if any upgrades are required, the central server 10 queries a ‘versions’ file located on the drug company computer system 200 and compares it against its own ‘versions’ file, which tracks the current versions of all software applications, operating systems, and database structures. The central server 10 could assign a unique version ID for each application and file within the ‘versions’ file for this purpose. Alternatively, using Microsoft Windows, the versions of the software applications, operating systems, and database structures located on the drug company computer system 200 could be identified by the “Properties” attribute of the application or file or the “Last Modified Date” of each application or file. If there are no updates required, the drug company computer system 200 disconnects from the central server 10 (process block 899). If there are updates required, the central server 10 determines what updates are required and uploads the required updates to the drug company computer system 200, which then installs the updates.

If the central server 10 determines that updates are required, it first queries the drug company computer system 200 to determine what software application(s), if any, need to be updated, as shown in process block 855. If there are software updates required, they are uploaded to the drug company computer system 200 and installed, as shown in process block 860. Once the software updates have been uploaded and installed, or if there are no software updates required, the central server 10 queries the drug company computer system 200 to determine what operating system updates, if any, are required, as shown in process block 865. If there are operating system updates required, they are uploaded to the drug company computer system 200 and installed, as shown in process block 870. Once the operating system updates have been uploaded and installed, or if there are no operating system updates required, the central server 10 queries the drug company computer system 200 to determine what database structure(s), if any, need to be updated, as shown in process block 875. If there are database structure updates required, they are uploaded to the drug company computer system 200 and installed, as shown in process block 880. Once the database structure updates have been uploaded and installed, or if there are no database structure updates required, the drug company computer system 200 disconnects from the central server 10 and reboots, as shown in process block 885. All of the updates transmitted from the central server 10 to the drug company computer system 200 are encrypted by the encryption software 70, transmitted over the communications network 60, and decryption using the encryption software 280 once received.

Once the drug company computer system 200 completes its reboot, it again connects to the central server 10 through the communications network 60 as described above, as shown in process block 887. Once the drug company computer system 200 has reconnected to the central server 10, the central server 10 queries the drug company computer system 200, as described above, to verify that all of the updates were successful, as shown in process block 890. If all of the updates were successful, the central server 10 logs that all updates were successful, as shown in process block 893, and disconnects. If all of the updates were not successful, the central server 10 logs which updates were not successful, as shown in process block 896, and checks if there have been three unsuccessful updates, as shown in process block 897. If there have not been three unsuccessful updates, the update procedure for the unsuccessful updates is repeated by returning to process block 860, 870, or 800, as required. If there have been three unsuccessful updates, or if the drug company computer system 200 fails to reboot and reconnect to the central server 10, the central server 10 generates a trouble report, which assigns a technician to be dispatched to the drug company for further diagnostics and/or repair. If the drug company computer system 200 is self-maintained by the drug company, a trouble report is generated by the central server 10 and communicated to the drug company, such as by email, fax, telephone, etc.

The foregoing description of the preferred embodiments of the invention have been presented for purposes of illustration and description, and are not intended to be exhaustive or to limit the invention to the precise forms disclosed. The descriptions were selected to best explain the principles of the invention and their practical application to enable other skills in the art to best utilize the invention in various embodiments and various modifications as are suited to the particular use contemplated. It is intended that the scope of the invention not be limited by the specification, but be defined by the claims set forth below.

I claim:

1. A system for drug sample inventory and tracking, comprising:
a) a central server, having a memory that stores information that includes drug sample information, which provides detailed drug sample information for drug samples that are delivered to and dispensed from a remote medical center, and that stores drug sample tracking information, which provides information on the drug samples delivered to the remote medical center and the drug samples dispensed from the remote medical center, and having communications means for coupling the drug sample information and drug sample tracking information between the central server and the remote medical center; and
b) a medical center computer system, located at the remote medical center, the medical center computer system having means for entering and storing the drug sample tracking information, means for indicating the inventory of drug samples, and second communications means for coupling drug sample information between the medical center computer system and the central server and coupling drug sample tracking information to the central server.

2. A system for drug sample inventory and tracking, as recited in claim 1, in which the medical center computer system includes means for entering patient information and means for coupling the patient information to the central server; and the central server memory stores the patient information, which includes information about patients to which drug samples are dispensed.

3. A system for drug sample inventory and tracking, as recited in claim 2, in which the means for coupling the patient information includes means for encrypting the patient information.

4. A system for drug sample inventory and tracking, as recited in claim 2, in which the medical center computer system includes a means for storing the patient information.

5. A system for drug sample inventory and tracking, as recited in claim 1, in which the central server memory stores user information, which indicates those users who are authorized to use the system; the central server includes means for coupling the user information to the medical center computer system; and the medical center computer system includes means for verifying that a user is authorized to use the system based on the user information received from the central server.

6. A system for drug sample inventory and tracking, as recited in claim 2, in which the patient information stored at the central server memory includes a patient identifier, a patient name, a patient age, a patient gender, a patient symptom, a patient diagnosis, a drug sample identifier for a drug sample dispensed to the patient, and a result of the drug sample dispensed to the patient.

7. A system for drug sample inventory and tracking, as recited in claim 1, in which the drug sample information stored in the central server memory includes a drug sample identifier, a drug sample name, a drug company name, an expiration date, a dosage, and a quantity.

8. A system for drug sample inventory and tracking, as recited in claim 1, in which the drug sample tracking information stored in the central server memory includes a drug sample identifier, a drug sample name, a drug company name, a medical center identifier for the medical center that dispensed the drug sample, a drug sample dispensing history, and an inventory of the drug sample at the medical center.

9. A system for drug sample inventory and tracking, as recited in claim 1, in which the first and second communications means include means for coupling data between the central server and the medical center computer system using the Internet.

10. A system for drug sample inventory and tracking, as recited in claim 9, in which the medical center computer system includes a processor and the second communications means includes a web browser program that is executed by the processor.

11. A system for drug sample inventory and tracking, as recited in claim 1, in which the medical center computer system includes a memory for storing the drug sample information, for storing the drug sample tracking information, and for storing drug sample inventory information at the remote medical center; and the first and second communications means operate to periodically couple the drug sample information and the drug sample tracking information between the central server memory and the medical center computer system memory.

12. A system for drug sample inventory and tracking, as recited in claim 11, in which the medical center computer system memory and the central server memory also store patient information, which contains information about patients to which drug samples are dispensed; the medical center computer system also includes means for entering the patient information; and the medical center computer system also includes means for periodically communicating the patient information to the central server.

13. A system for drug sample inventory and tracking, as recited in claim 12, in which the means for communicating the patient information includes means for encrypting the patient information.

14. A system for drug sample inventory and tracking, as recited in claim 11, in which the central server memory also stores user information, which indicates which users are authorized to use the system; the central server also includes means for coupling the user information to the medical center computer system; the medical center computer system memory stores the user information; and the medical center computer system includes means for verifying that a user is authorized to use the system based on the user information stored in the medical center computer system memory.

15. A system for drug sample inventory and tracking, as recited in claim 12, in which the stored patient information includes a patient identifier, a patient name, a patient age, a patient gender, a patient symptom, a patient diagnosis, a drug sample identifier for a drug sample dispensed to the patient, and a result of the drug sample dispensed to the patient.

16. A system for drug sample inventory and tracking, as recited in claim 11, in which the stored drug sample information includes a drug sample identifier, a drug sample name, a drug company name, an expiration date, a dosage, and a quantity.

17. A system for drug sample inventory and tracking, as recited in claim 11, in which the stored drug sample tracking information includes a drug sample identifier, a drug sample name, a drug company name, a medical center identifier for the medical center that dispensed the drug sample, a history of the drug samples dispensed from the medical center, and an inventory of drug samples at the medical center.

18. A system for drug sample inventory and tracking, as recited in claim 11, in which the first and second communications means include means for coupling data between the central server and the medical center computer system using the Internet.

19. A system for drug sample inventory and tracking, as recited in claim 18, in which the medical center computer system includes a processor and the second communications means includes a web browser program that is executed by the processor.

20. A system for drug sample inventory and tracking, as recited in claim 1, further comprising a drug company computer system, located at a remote drug company facility, the drug company computer system including: means for entering drug sample information and a third communications means for coupling drug sample information to the central server and for coupling drug sample tracking information from the central server.

21. A system for drug sample inventory and tracking, as recited in claim 20, in which the central server memory stores patient information, which contains information about patients to which drug samples are dispensed; the medical center computer system includes means for entering the patient information and means for coupling the patient information to the central server memory; and the central server also includes means for coupling the stored patient information to the drug company computer system.

22. A system for drug sample inventory and tracking, as recited in claim 21, in which the means for coupling the patient information includes means for encrypting the patient information.

23. A system for drug sample inventory and tracking, as recited in claim 20, in which the central server memory stores user information, which indicates users who are authorized to use the system; the central server also includes means for coupling the user information to the drug company computer system; and the drug company computer system includes means for verifying that a user is authorized to use the system based on the user information received from the central server.

24. A system for drug sample inventory and tracking, as recited in claim 21, in which the patient information stored at the central server memory includes a patient identifier, a patient name, a patient age, a patient gender, a patient symptom, a patient diagnosis, a drug sample identifier for a drug sample dispensed to the patient, and a result of the drug sample dispensed to the patient.

25. A system for drug sample inventory and tracking, as recited in claim 20, in which the drug sample information stored at the central server memory includes a drug sample identifier, a drug sample name, a drug company name, an expiration date, a dosage, and a quantity.

26. A system for drug sample inventory and tracking, as recited in claim 20, in which the drug sample tracking information stored at the central server memory includes a drug sample identifier, a drug sample name, a drug company name, a medical center identifier for the medical center that dispensed the drug sample, a history of the drug samples dispensed from the medical center, and an inventory of the drug samples at the medical center.

27. A system for drug sample inventory and tracking, as recited in claim 20, in which the first and third communications means include means for coupling data between the central server and the drug company computer system using the Internet.

28. A system for drug sample inventory and tracking, as recited in claim 27, in which the drug company computer system includes a processor and the third communications means includes a web browser program that is executed by the processor.

29. A system for drug sample inventory and tracking, as recited in claim 20, in which the drug company computer system includes a memory for storing the drug sample information and for storing the drug sample tracking information; and the first and third communications means operate to periodically couple the drug sample information and the drug sample tracking information between the central server memory and the drug company computer system memory.

30. A system for drug sample inventory and tracking, as recited in claim 29, in which the central sever memory and the drug company computer system memory also store patient information, which contains information about patients to which drug samples are dispensed; and the drug company computer system also includes means for periodically retrieving information from the central server.

31. A system for drug sample inventory and tracking, as recited in claim 30, in which the means for communicating the patient information includes means for encrypting the patient information.

32. A system for drug sample inventory and tracking, as recited in claim 29, in which the central server memory also stores user information, which indicates which users are authorized to use the system; the central server also includes means for coupling the user information to the drug company computer system; the drug company computer system memory stores the user information; and the drug company computer system includes means for verifying that a user is authorized to use the system based on the user information stored in the drug company computer system memory.

33. A system for drug sample inventory and tracking, as recited in claim 30, in which the stored patient information includes a patient identifier, a patient name, a patient age, a patient gender, a patient symptom, a patient diagnosis, a drug sample identifier for a drug sample dispensed to the patient, and a result of the drug sample dispensed to the patient.

34. A system for drug sample inventory and tracking, as recited in claim 30, in which the stored drug sample information includes a drug sample identifier, a drug sample name, a drug company name, an expiration date, a dosage, and a quantity.

35. A system for drug sample inventory and tracking, as recited in claim 30, in which the stored drug sample tracking information includes a drug sample identifier, a drug sample name, a drug company name, a medical center identifier for the medical center that dispensed the drug sample, a history of the drug samples dispensed from the medical center, and an inventory of the drug samples at the medical center.

36. A system for drug sample inventory and tracking, as recited in claim 29, in which the first and second communications means includes means for coupling data between the central server and the drug company computer system using the Internet.

37. A system for drug sample inventory and tracking, as recited in claim 36, in which the drug company computer system includes a processor and the second communications means includes a web browser program that is executed by the processor.

38. A system for drug sample inventory and tracking, as recited in claims 1, 11, 20, or 29 in which the drug sample tracking information includes information on drug samples from drug companies that do not use the system that are delivered to the remote medical center and dispensed from the remote medical center.

39. A method for drug sample inventory and tracking, comprising the steps of:
connecting a central server to a network;
storing in a memory on the central server drug sample information, which provides detailed drug sample information for drug samples that are delivered to and dispensed from a remote medical center;
connecting a medical center computer system, located at the remote medical center, to the network;
entering at the medical center computer system drug sample tracking information, which includes information on the drug samples delivered to the remote medical center, information on the drug samples dispensed from the remote medical center, and information on the inventory of drug samples at the remote medical center;
communicating the drug sample information between the central server and the medical center computer system through the network;
communicating the drug sample tracking information from the medical center computer system to the central server through the network; and
storing the drug sample tracking information and drug sample inventory information in the central server memory.

40. A method for drug sample inventory and tracking, as recited in claim 39, further comprising the steps of:
entering patient information at the medical center computer system, which includes information about patients to which drug samples are dispensed;
communicating the patient information from the medical center computer system to the central server through the network; and
storing the patient information in the central server memory.

41. A method for drug sample inventory and tracking, as recited in claim 40, in which communicating the patient information from the medical center computer system to the central server includes encrypting the patient information.

42. A method for drug sample inventory and tracking, as recited in claim 40, further including the step of storing the patient information in a memory on the medical center computer system.

43. A method for drug sample inventory and tracking, as recited in claim 39, further comprising the steps of:
storing user authorization information in the central server memory; and
communicating the user authorization information from the central server to the medical center computer system through the network.

44. A method for drug sample inventory and tracking, as recited in claim 39, further comprising the steps of:
storing in a memory on the medical center computer system the drug sample information, the drug sample tracking information, and drug sample inventory information; and
periodically connecting the central server and the medical center computer system to the network to download the drug sample tracking information stored in the medical center computer system memory to the central server memory and to synchronize the drug sample information in the medical center computer system memory with the drug sample information in the central server memory.

45. A method for drug sample inventory and tracking, as recited in claim 44, further comprising the steps of:
entering patient information at the medical center computer system, which includes information about patients to which drug samples are dispensed;
storing the patient information in the medical center computer system memory; and
communicating the patient information stored in the medical center computer system memory to the central server memory when the central server and the medical center computer system periodically connect to the network.

46. A method for drug sample inventory and tracking, as recited in claim 45, in which the step of communicating the patient information stored in the medical center computer system memory to the central server memory includes encrypting the patient information.

47. A method for drug sample inventory and tracking, as recited in claim 44, further comprising the steps of:
storing user authorization information in the central server memory;
storing the user authorization information in the medical center computer system memory; and
synchronizing the user authorization information in the central server memory and the user authorization information in the medical center computer system memory when the central server and the medical center computer system periodically connect to the network.

48. A method for drug sample inventory and tracking, as recited in claims 39 or 40, further comprising the steps of:
uploading a software update from the central server to the medical center computer system; and
installing the software update on the medical center computer system.

49. A method for drug sample inventory and tracking, as recited in claims 39 or 40, further comprising the steps of:
uploading an operating system update from the central server to the medical center computer system; and
installing the operating system update on the medical center computer system.

50. A method for drug sample inventory and tracking, as recited in claims 39 or 40, further comprising the steps of:
uploading a database structure update from the central server to the medical center computer system; and
installing the database structure update on the medical center computer system.

51. A method for drug sample inventory and tracking, as recited in claim 39, further comprising the steps of:
connecting a drug company computer system, located at a remote drug company facility, to the network;
entering the drug sample information at the drug company computer system;
communicating the drug sample information from the drug company computer system to the central server through the network; and
communicating the drug sample tracking information from the central server to the drug company computer system through the network.

52. A method for drug sample inventory and tracking, as recited in claim 51, further comprising the steps of:
storing patient information, which provides information about patients to which drug samples are dispensed, in the central server memory; and
communicating the patient information from the central server to the drug company computer system through the network.

53. A method for drug sample inventory and tracking, as recited in claim 52, in which communicating the patient information from the central server to the drug company computer system includes encrypting the patient information.

54. A method for drug sample inventory and tracking, as recited in claim 51, further comprising the steps of:
storing user authorization information in the central server memory; and
communicating the user authorization information from the central server to the drug company computer system through the network.

55. A method for drug sample inventory and tracking, as recited in claim 51, further comprising the steps of:
storing in a memory on the drug company computer system the drug sample information and the drug sample tracking information; and
periodically connecting the central server and the drug company computer system to the network, to communicate the drug sample information stored in the drug company computer system memory to the central server memory and to download the drug sample tracking information from the central server memory to the drug company computer system memory.

56. A method for drug sample inventory and tracking, as recited in claim 55, further comprising the steps of:
storing in the central server memory patient information, which includes information about patients to which drug samples are dispensed,; and
downloading the patient information stored in the central server memory to the drug company computer system memory when the central server and the drug company computer system periodically connect to the network.

57. A method for drug sample inventory and tracking, as recited in claim 56, in which the step of downloading the patient information stored in the central server memory to the drug company computer system memory comprises encrypting the patient information.

58. A method for drug sample inventory and tracking, as recited in claim 55, further comprising the steps of:
storing in the central server memory user authorization information;
storing the user authorization information in the drug company computer system memory; and
synchronizing the user authorization information in the central server memory and the user authorization information in the drug company computer system memory when the central server and the drug company computer system periodically connect to the network.

59. A method for drug sample inventory and tracking, as recited in claims 51 or 52, further comprising the steps of:
uploading a software update from the central server to the drug company computer system; and
installing the software update on the drug company computer system.

60. A method for drug sample inventory and tracking, as recited in claims 51 or 52, further comprising the steps of:
uploading an operating system update from the central server to the drug company computer system; and
installing the operating system update on the drug company computer system.

61. A method for drug sample inventory and tracking, as recited in claims 51 or 52, further comprising the steps of:
uploading a database structure update from the central server to the drug company computer system; and
installing the database structure update on the drug company computer system.

62. A system for drug sample inventory and tracking, as recited in claim 20, in which the central server memory stores subscriber services information, which includes information on each medical center and drug company that uses the system.

63. A system for drug sample inventory and tracking, as recited in claim 62, in which the subscriber services information includes an indication that patient information will be provided by the medical center computer system.

64. A system for drug sample inventory and tracking, as recited in claim 62, in which the subscriber services information includes an indication that patient information should be downloaded to the drug company computer system.

65. A system for drug sample inventory and tracking, as recited in claim 62, in which the subscriber services information includes an indication that non-customer drug sample information should be downloaded to the drug company computer system.

66. A system for drug sample inventory and tracking, as recited in claims 1, 2, 11, or 12, in which the medical center computer system includes means for checking an expiration date for the drug samples.
